(19) United States
(12) Patent Application Publication
(10) Pub. No.: US 2023/0130371 A1
(43) Pub. Date: Apr. 27, 2023

(54) RNA Detection by Selective Labeling and Amplification

(71) Applicant: Akoya Biosciences, Inc., Menlo Park, CA (US)

(72) Inventors: Peter J. Miller, Cambridge, MA (US); Julia Kennedy-Darling, Redwood City, CA (US)

(21) Appl. No.: 17/880,323

(22) Filed: Aug. 3, 2022

Related U.S. Application Data
(60) Provisional application No. 63/229,064, filed on Aug. 3, 2021.

(51) Int. Cl. C12Q 1/6818

(52) U.S. Cl. CPC C12Q 1/6818

(57) ABSTRACT

Methods for labeling biological samples include (a) contacting a biological sample featuring a target RNA with a probe, where the probe includes a capture moiety that specifically binds to the target RNA, and a plurality of reporter moieties, and (b) for each reporter moiety of the plurality of reporter moieties: contacting the reporter moiety with a catalytic agent that includes an oligonucleotide that specifically binds to the reporter moiety, and a reactive species linked to the oligonucleotide, and contacting the biological sample with a labeling agent that reacts with the reactive species to deposit the labeling agent or a derivative thereof in the sample in proximity to the target RNA.

### CROSS-REFERENCE TO RELATED APPLICATIONS

This application claims priority to U.S. Provisional Patent Application No. 63/229,064, filed on Aug. 3, 2021, the entire contents of which are incorporated herein by reference.

### TECHNICAL FIELD

This disclosure relates to the detection of biological analytes in a sample by selective labeling.

### BACKGROUND

Oligonucleotide probes have been used to detect nucleic acid target analytes such as DNA and RNA species in tissue samples. Fluorescent probes can be used to directly detect specific target analytes. In general, the number of different target analytes that can be detected depends upon the number of different fluorescent signals that can be reliably distinguished.

### SUMMARY

This disclosure features methods for selectively applying dyes and other labeling species to samples to identify and quantify specific RNA analytes in the samples. Multiple dyes and labeling agents can be added to the sample serially or in parallel to identify and quantify a variety of target RNAs in one or more detection cycles.

The methods can be performed by binding multiple probes to a sample, where each probe includes a capture moiety (e.g., an oligonucleotide sequence) that specifically binds to a target RNA species in the sample, and a set of oligonucleotide sequences that are linked to or contiguous with the capture moiety. A catalytic is then introduced, and includes an oligonucleotide linked to a reactive species. The oligonucleotide of the catalytic agent hybridizes to one of the members of the set of oligonucleotide sequences of the probe, localizing the catalytic agent in the sample at positions corresponding to the target RNA. A reaction between the reactive species and a labeling agent that is introduced deposits the labeling agent in proximity to that target RNA. The catalytic agent can then be removed by dehybridization under relatively mild conditions, ensuring that each of the probes remains bound to the sample. The procedure is repeated with additional catalytic agents, each of which includes an oligonucleotide that binds to a different member of the set of oligonucleotide sequences of the probe, coupled to a reactive species. Reactions between the reactive species of each of the catalytic agents and different labeling agents deposit each of the different labeling agents in proximity to the target RNA. In such a manner, populations of each of the different labeling agents are located near the target RNA in the sample.

The foregoing procedure can be performed simultaneously at multiple locations in the sample, and for multiple different probes that bind to different target RNAs. In this manner, each of the multiple different target RNAs can be labeled with different combinations of populations of labeling agents. The relatively mild conditions under which the catalytic agents are removed from the sample ensures that the probes remain bound to the sample, and sample integrity is maintained.

The disclosure features methods that include (a) contacting a biological sample featuring a target RNA with a probe, where the probe includes a capture moiety that specifically binds to the target RNA, and a plurality of reporter moieties, and (b) for each reporter moiety of the plurality of reporter moieties: contacting the reporter moiety with a catalytic agent featuring an oligonucleotide that specifically binds to the reporter moiety, and a reactive species linked to the oligonucleotide; and contacting the biological sample with a labeling agent that reacts with the reactive species to deposit the labeling agent or a derivative thereof in the sample in proximity to the target RNA.

Embodiments of the methods can include any one or more of the following features.

The probe can include at least three different reporter moieties (e.g., at least four different reporter moieties). The catalytic agent can include an enzyme. The enzyme can include horseradish peroxidase. The labeling agent can include an enzyme substrate. The enzyme substrate can include an inactive tyramide species. The labeling agent can include an enzyme substrate conjugated to an oligonucleotide.

The methods can include repeating steps (a) and (b) for additional target RNAs in the biological sample. For each different type of target RNA in the biological sample, a unique combination of different labeling agents can be deposited in the biological sample in proximity to target RNA molecules. Each of the different labeling agents can include an oligonucleotide featuring a unique sequence.

The methods can include: (c) exposing the biological sample to a plurality of optical labels, where each of the optical labels features an oligonucleotide having a sequence, and a species that generates an optical signal; (d) measuring optical signals generated by optical labels of the plurality of optical labels that hybridize to complementary oligonucleotides of the labeling agents; (e) repeating steps (c) and (d) with different pluralities of optical labels; (f) identifying one or more of the unique combinations of different labeling agents in the biological sample based on the measured optical signals; and (g) determining a location of one or more of the target RNAs in the biological sample based on the identified combinations of different labeling agents.

The methods can include, for at least one of the optical labels, removing the at least one of the optical labels from the biological sample after measuring an optical signal generated by the at least one of the optical labels and before repeating steps (c) and (d) with different pluralities of optical labels. Removing the at least one of the optical labels can include dehybridizing the at least one of the optical labels from oligonucleotides of one or more of the labeling agents.

Each unique combination of different labeling agents can include a same number of different labeling agents. The species that generates the optical signal can be a fluorescent moiety. The species that generates the optical signal can include at least one fluorescent nucleotide.

Measuring optical signals generated by the optical labels can include obtaining at least one image of the optical labels in the biological sample, and identifying optical signals corresponding to the optical labels in the at least one image. Each plurality of optical labels in step (c) can include a same number of different types of optical labels. Each plurality of optical labels in step (c) can include 3 or more (e.g., 5 or more) different types of optical labels.

The methods can include repeating step (c) until the biological sample has been exposed to a set of optical labels, where each labeling agent deposited in the biological sample has a complementary optical label in the set of optical labels. The methods can include exposing the biological sample to at least one of the plurality of optical labels more than once. Each time step (c) is performed, each member of the plurality of optical labels in step (c) can include a species that generates a different optical signal. The different optical signals can have different spectral distributions.

Among the plurality of optical labels, at least two of the optical labels can include a common species that generates the optical signal, and the at least two of the optical labels can be exposed to the biological sample during different repetitions of step (c). Among the plurality of optical labels, first and second optical labels can each include a first species that generates the optical signals of the first and second optical labels, third and fourth optical labels can each include a second species that generates the optical signals of the third and fourth optical labels, and the first and second species can be different. The optical signals generated by the first and second species can be different. The methods can include exposing the sample to the first and second optical labels during different repetitions of step (c), and exposing the sample to the third and fourth optical labels during different repetitions of step (c).

The labeling agent can include a species that generates an optical signal. The species that generates the optical signal can be a fluorescent moiety.

The methods can include obtaining one or more images of the sample that include contributions from each of the labeling agents, where different labeling agents are associated with each of the reporter moieties of the plurality of reporter moieties. The methods can include identifying the target RNA in the biological sample based on the contributions from each of the labeling agents in the one or more images of the sample.

The methods can include repeating steps (a) and (b) for multiple different target RNAs in the biological sample. The methods can include, prior to repeating steps (a) and (b), inactivating at least some of the deposited labeling agents in the sample.

The methods can include selecting the labeling agents so that a different combination of labeling agents is deposited in the biological sample in proximity to at least some of the different target RNAs, relative to a combination of labeling agents deposited in the biological sample in proximity to at least some others of the different target RNAs. The methods can include selecting the labeling agents so that a unique combination of labeling agents is deposited in the biological sample in proximity to each different target RNA. At least some unique combinations of labeling agents can include three or more (e.g., four or more, five or more) different labeling agents. Each unique combination of labeling agents can include four or more different labeling agents. Each unique combination of labeling agents can include a same number of labeling agents.

The one or more images can include contributions from labeling agents associated with only one type of target RNA in the biological sample. The one or more images can include contributions from labeling agents associated with more than one type of target RNA in the biological sample.

Embodiments of the methods can also include any of the other features described herein, and can include any combination of features that are individually described in connection with different embodiments, except as expressly stated otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1 is a schematic diagram showing a series of example steps for labeling of RNA in a biological sample.

FIGS. 2A-2E are schematic diagrams showing example steps for identifying RNA in a biological sample.

FIGS. 3A-3D are schematic diagrams showing examples of oligonucleotides.

FIGS. 4A-4C are schematic diagrams showing examples of probes.

FIG. 4D is a schematic diagram showing an example of a catalytic agent.

FIG. 4E is a schematic diagram showing an example of a probe.

FIG. 5 is a flow chart showing a series of example steps for analysis of a biological sample.

FIG. 6 is a flow chart showing another series of examples steps for analysis of a biological sample.

FIG. 7A is a schematic diagram showing an example set of detection cycles for identifying an analyte in a sample.

FIG. 7B is a schematic diagram showing two different types of RNAs in a sample and corresponding probes that hind to the analytes.

FIG. 7C is a schematic image of the sample of FIG. 7B showing locations of the two different types of RNAs in the sample.

FIG. 7D is a schematic diagram showing optical signals measured in each of 6 detection cycles performed on a sample containing both of the different RNAs of FIG. 7B.

FIG. 8 is a schematic diagram showing an example multispectral imaging system.

FIG. 9 is a schematic diagram showing an example controller.

FIG. 10 is a schematic diagram of a system for analyzing a biological sample.

Like reference symbols in the various drawings indicate like elements.

### DETAILED DESCRIPTION

### Introduction

Analysis of multiple target RNA analytes in a biological sample is an important aspect of modern research methods. Detecting localized expression of different RNA species provides important information about cellular regulation, function, disease progression, and treatment efficacy.

Conventional techniques for detecting RNA species include fluorescence in-situ hybridization, in which a probe that includes a nucleotide sequence that is complementary to an RNA target analyte is introduced, and selectively hybridizes to the RNA target analyte. The probe can include fluorescent nucleotides that generate a measurable signal for detection of the bound probe in the sample.

One of difficulties of applying such methods to the detection of RNA analytes is the extent to which multiplexing of the methods is limited. Because each RNA analyte is essentially associated with a distinct fluorescence signal (e.g., a fluorescence signal at a different wavelength), analysis of different RNA analytes involves separation of different measured fluorescence signals. There are practical limits on viability of optical and computational methods for separating and accurately quantifying such signals that effectively limit the application of these methods to a relatively small number of different RNA analytes.

Another difficult associated with such methods is 1:1 relationship between the probe and the RNA analyte to which it binds. Because the probe contains the signal-generating fluorophore(s), the measured fluorescence signals are limited in intensity to the number of fluorophores present in each probe, in particular since each RNA analyte typically hybridizes to only one probe molecule. As a result, measured signals can be relatively weak, making the detection of RNA analytes challenging, particular in samples of comparatively poorer quality and/or in the presence of other probes generating fluorescence emission signals that overlap spectrally.

This disclosure features methods for performing multiplexed labeling, signal amplification, and quantification of target RNA analytes in a biological sample. The methods involve multiple cycles of introduction and removal of certain agents involved in the labeling process, without disrupting previously-deposited labels in the sample. Instead, removal of agents involved in the labeling process is performed by dehybridizing the agents under relatively mild conditions, preserving sample integrity and ensuring that removal of the agents is nearly complete. As a result, little or no cross-species labeling occurs. Instead, each target RNA analyte in the sample can be selectively labeled with a different combination of multiple labeling species, and deposition of each labeling species is highly constrained to regions of the sample that correspond to specific target RNA analytes.

### General Methodology

This disclosure describes a variety of different analytical methodologies for identifying and quantifying multiple target RNA analytes in a biological sample. As described above, in conventional fluorescence imaging methods, the number of RNA analytes that can be detected can be limited by the number of different measurement signals that can be detected and resolved. Methods exist for separating fluorescence emission signals from dyes that overlap spectrally, even when the overlap is significant. For example, eigenvector decomposition methods such as spectral unmixing can be used to separate such signals, provided that relatively good estimates for the pure spectra of the individual fluorescent dyes (i.e., the eigenvectors) are available or measurable. Even with such techniques, however, the number of different measurement signals that can be resolved—and the corresponding number of different RNA analytes that can be quantified—may be relatively limited due to spectral congestion of the dyes involved.

Limits on the number of different analytes may not represent significant drawbacks for certain types of assays. For example, for certain tumor-specific assays that target a relatively small number of expressed cell markers via antibody-based probes that selectively bind to the markers, the foregoing limits may not represent a significant shortcoming. However, nucleic acid assays that target cellular RNA and DNA may be designed to quantify tens, hundreds, or thousands of distinct transcripts. For these expansive assays, the above limitations may represent significant roadblocks.

One approach to increasing the number of distinct RNA analytes that can be detected and quantified involves using reporter moieties with spectrally narrow and distinct emission characteristics. For example, quantum dot-based reporter moieties can be synthesized and incorporated into probes that bind specifically to different types of analytes. By controlling the composition and size distribution of the quantum dots, populations of different reporter moieties with (spectrally) closely-spaced but resolvable fluorescence emission signals can be obtained. Incorporated into different types of probes, the resolvable reporter moieties can be used to identify a larger number of different analytes than would typically be possible with conventional fluorescent dye-based reporter moieties.

However, as the number of different analytes increases, the complexity associated with synthesizing populations of distinct reporter moieties increases, as does the time required to synthesize and characterize the reporter moieties prior to use. For an assay involving several hundred different RNA analytes, the synthetic burdens discussed above may be too significant to make such approaches practical and cost-effective.

The methods described herein can be used to detect a wide variety of different RNA analytes in a sample. Instead of assigning a different “color” to each type of RNA analyte via a distinct reporter moiety that is specifically bound only to RNAs of that type, the methods described herein involve labeling individual RNAs with combinations of different optical labels. Each different type of RNA is labeled with a distinct combination of optical labels that constitute a reporter code. Although individual optical labels may be present in reporter codes that correspond to different types of RNAs, each type of RNA is associated with a different reporter code (i.e., a different, unique combination of optical labels). By detecting optical signals generated by the optical labels and determining which optical labels are co- localized in the sample, the locations of specific reporter codes in the sample can be determined, and the spatial locations of different types of RNAs in the sample can be elucidated.

By labeling RNAs with combinations of optical labels, comparatively fewer individually distinct optical labels are needed to distinguish among a set of different RNA analytes. As a result, the spectral congestion and difficulties associated with resolving spectrally closely-spaced emission signals can be substantially reduced. Consequently, a much larger number of different RNAs can be reliably detected and quantified. Further, existing sets of probes that are designed to target particular groups of RNAs can be more readily augmented to include probes for additional RNAs without resorting to complex synthetic methods to produced tightly controlled distributions of spectrally distinct fluorescence probes.

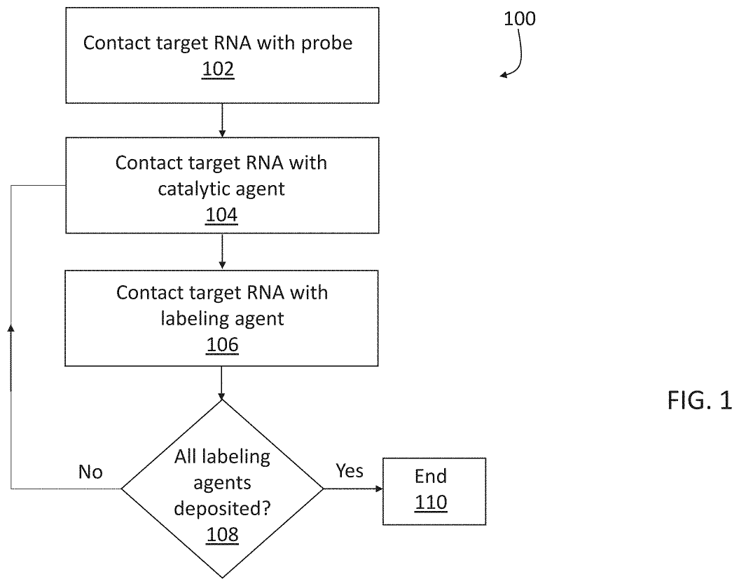

FIG. 1 is a flow chart 100 showing a series of example steps for implementing one method of sample analysis. In a first step 102, a target RNA is contacted with a first agent that specifically binds to the target RNA.

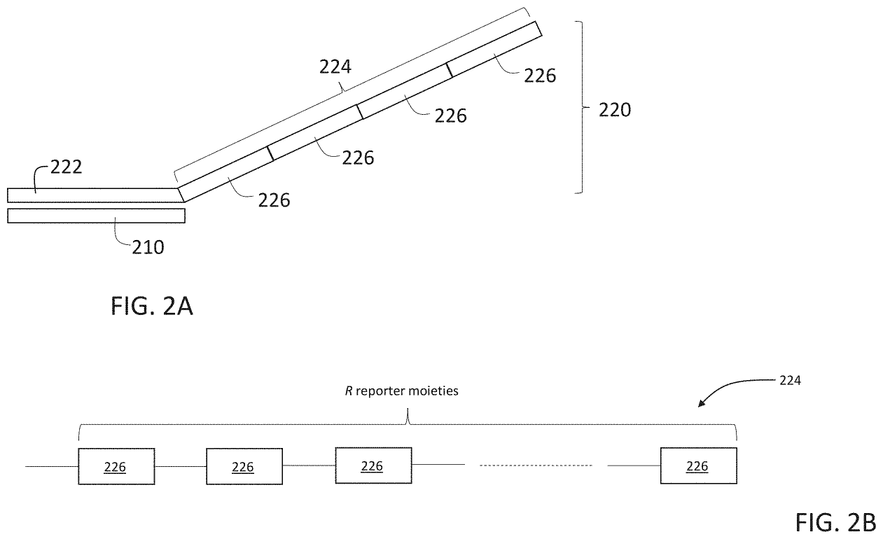

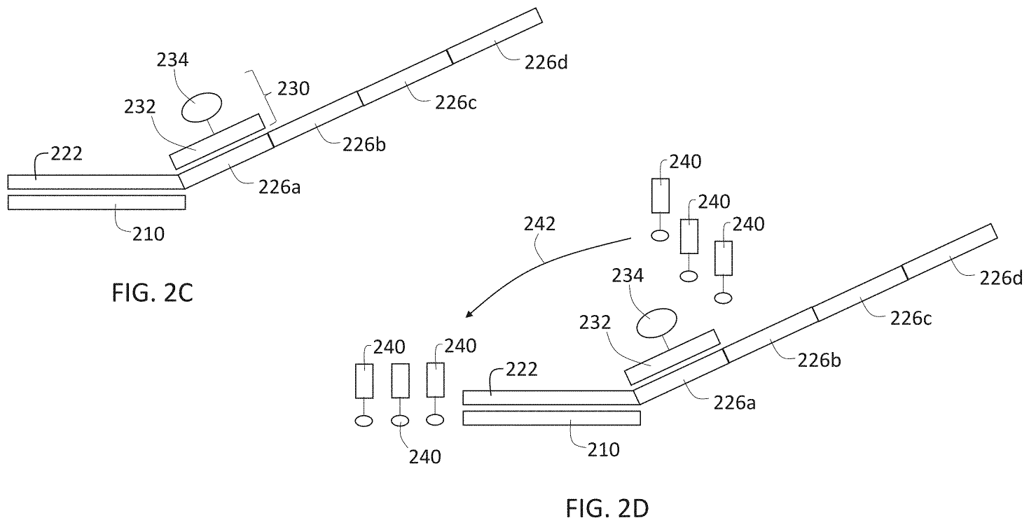

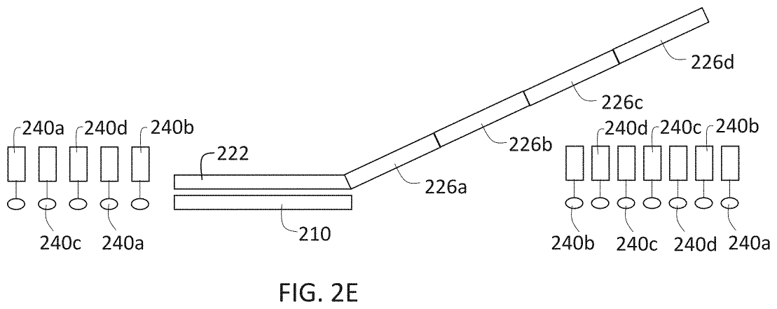

This first step is illustrated schematically in FIG. 2A. In FIG. 2A, a target RNA 210 in a biological sample is contacted with a probe 220. Probe 220 includes a capture moiety 222 that specifically binds to target RNA 201, and a detection moiety 224 that is linked to capture moiety 222. In this manner, probe 220 specifically localizes at positions in the sample that correspond to target RNA 210.

As used herein, the terms “contacts” and “contacting” mean that an agent, species, moiety, or other element is brought into association with a sample, or another agent, species, moiety, or element, such that the two interact with one another. For example, when one entity is “contacted” with a second entity as described herein, the second entity is brought into close enough association with the first entity that they interact, and can bind, associate, react, or otherwise undergo a transformation as a result of the interaction.

Target RNA 210 can be located in a biological sample, which can be any one of a variety of different types of samples. Examples of biological samples include, but are not limited to, tissue sections (e.g., fresh sections, fresh-frozen sections, formalin-fixed paraffin embedded sections), tissue biopsies, cells, cell suspensions, cell dispersions, cell cultures, and various bodily fluids such as blood, urine, interstitial fluid, and lymphatic fluid.

The biological sample can be immobilized on a surface. For example, the surface can be a surface of a slide, a plate, a well, a tube, a membrane, or a film. In some embodiments, the biological sample can be mounted on a slide. In certain embodiments, the biological sample can be fixed using a fixative, such as an aldehyde, an alcohol, an oxidizing agent, a mercurial, a picrate, HOPE fixative, or another fixative. The biological sample may alternatively, or in addition, be fixed using heat fixation. Fixation can also be achieved via immersion or perfusion.

Target RNA 210 can be any of a variety of different types of RNA. Examples of such RNA types include, but are not limited to, messenger RNA, ribosomal RNA, transfer RNA, transfer-messenger RNA, small nuclear RNA, small nucleolar RNA, signal recognition particle RNA, guide RNA, SmY RNA, Y RNA, antisense RNA, CRISPR RNA, microRNA, small interfering RNA, short hairpin RNA, long noncoding RNA, and enhancer RNA. RNA 20 can contain RNA bases, both DNA and RNA bases, and synthetic bases.

As shown in FIG. 2A, probe 220 includes a capture moiety 222 and a detection moiety 224 linked to capture moiety 102. Capture moiety 222 selectively binds to RNA 210 to attach detection moiety 224 to RNA 210. A variety of different reversible and irreversible binding mechanisms can occur between capture moiety 222 and RNA 210. In some embodiments, for example, capture moiety 222 includes an oligonucleotide having a sequence that is at least partially complementary to a sequence of RNA 210. When RNA 210 is exposed to probe 220, capture moiety 222 binds to RNA 210 by hybridizing to RNA 210. The binding between capture moiety 222 and RNA 210 can be readily be reversed via dehybridization, e.g., by heating sample 10 and/or introducing one or more chaotropic reagents. In certain embodiments, where capture moiety 222 includes an oligonucleotide, the oligonucleotide includes 10 or more (e.g., 15 or more, 20 or more, 25 or more, 30 or more, 35 or more, 40 or more, 45 or more, 50 or more, 60 or more, 70 or more, 80 or more, 100 or more, 120 or more, 150 or more, 200 or more, 300 or more, 400 or more, 500 or more, 600 or more, 800 or more, 1000 or more, or even more) nucleotides.

As shown in FIG. 2A, capture moiety 222 is linked to detection moiety 224 in probe 220. The linkage between capture moiety 222 and detection moiety 224 can be implemented in various ways. In some embodiments, capture moiety 222 and detection moiety 224 can be linked directly via a covalent or non-covalent bond. That is, capture moiety 222 and detection moiety 224 can be linked directly via a bond, with no intervening moiety or structure between capture moiety 222 and detection moiety 224.

In certain embodiments, capture moiety 222 and detection moiety 224 can be linked via a primary-secondary antibody pair. To label RNA 210 with detection moiety 224, RNA 210 is exposed to a first labeling agent that includes capture moiety 222 conjugated to a primary antibody, which functions as a part of the linkage between capture moiety 222 and detection moiety 224. Once the first labeling agent selectively binds to RNA 210, a second labeling agent is introduced that includes detection moiety 224 conjugated to a secondary antibody that functions as another part of the linkage. The secondary antibody selectively binds to the first antibody, forming a linkage between capture moiety 222 and detection moiety 224 consisting of the associated antibodies, and labeling RNA 210 with detection moiety 224.

In some embodiments, the linkage between capture moiety 222 and detection moiety 224 can be implemented as a double-stranded nucleic acid (e.g., hybridized nucleic acid strands that are at least partially complementary). To label RNA 210 with detection moiety 224, RNA 210 is exposed to a first labeling agent that includes capture moiety 222 linked to a first nucleic acid, which functions as a part of the linkage between capture moiety 222 and detection moiety 224. Once the first labeling agent selectively binds to RNA 210, a second labeling agent is introduced that includes detection moiety 224 linked to a second nucleic acid that functions as another part of the linkage. The second nucleic acid is at least partially complementary to the first nucleic acid, and selectively hybridizes to the first nucleic acid, labeling RNA 210 with detection moiety 224.

In some embodiments, the first nucleic acid can be a nucleic acid sequence that is contiguous with capture moiety 222. In other words, capture moiety 222 and the first nucleic acid can form a continuous nucleic acid sequence in which a portion of the nucleic acid sequence functions as capture moiety 222 (i.e., a capture region), and a portion of the nucleic acid sequence functions as the first nucleic acid (i.e., a linking nucleic acid sequence). The continuous nucleic acid sequence can be single-stranded or double-stranded.

In certain embodiments, the second nucleic acid can be a nucleic acid sequence that is contiguous with detection moiety 224. That is, the second nucleic acid and detection moiety 224 can form a continuous nucleic acid sequence in which a portion of the nucleic acid sequence functions as the second nucleic acid (i.e., a linking nucleic acid region), and a portion of the nucleic acid sequence functions as detection moiety 224 (i.e., a detection region that includes the label regions described herein that form detection moiety 224). The continuous nucleic acid sequence can be single-stranded or double-stranded.

In some embodiments, capture moiety 222 can be linked to the first nucleic acid through conjugation, e.g., capture moiety 222 can be covalently bonded to the first nucleic acid. Any of a wide variety of different linkages can be used to covalently bond capture moiety 222 and the first nucleic acid, as discussed below. In certain embodiments, detection moiety 224 can be linked to the second nucleic acid through covalent bonding, using any of the different linkages described herein.

In some embodiments, the first and second nucleic acids that function as portions of the linkage between capture moiety 222 and detection moiety 224 do not directly hybridize. Instead, the first and second nucleic acids each hybridize to a portion of a bridging oligonucleotide that includes nucleic acid sequences that are at least partially complementary to each of the first and second nucleic acids. Bridging oligonucleotides can be single-stranded, such that a portion of the bridging oligonucleotide hybridizes to the first nucleic acid and a portion of the bridging oligonucleotide hybridizes to the second nucleic acid. Bridging oligonucleotides can be partially double-stranded, with overhangs on one or both strands that hybridize to the first and second nucleic acids to form the linkage between capture moiety 222 and detection moiety 224.

Bridging oligonucleotides can be linear such that a single capture moiety is linked to a single reporter moiety. Alternatively, bridging oligonucleotides can be branched, and can include a single nucleic acid sequence that hybridizes to capture moiety 222, and multiple nucleic acid sequences that hybridize to detection moieties 224. As a result, a single capture moiety 222 can be linked to multiple detection moieties 224, allowing for amplification of optical signals that correspond to the RNA to which capture moiety 222 selectively binds.

In certain embodiments, the linkage between capture moiety 222 and detection moiety 224 can be implemented as any of a variety of aliphatic and/or aromatic linking species. Further, as discussed above, in some embodiments, capture moiety 222 can be covalently bonded to the first nucleic acid, either via a direct covalent bond, or via any of a variety of aliphatic and/or aromatic linking species. Further still, as discussed above, in certain embodiments, detection moiety 224 can be covalently bonded to the second nucleic acid, either via a direct covalent bond, or via any of a variety of aliphatic and/or aromatic linking species.

Examples of linking species that can be used to directly link capture moiety 222 and detection moiety 224, to link capture moiety 222 and the first nucleic acid, and/or to link detection moiety 224 and the second nucleic acid, include—but are not limited to—C₁₋₂₀ cyclic and non-cyclic alkyl species, C₂₋₂₀ cyclic and non-cyclic alkene species, C₂₋₂₀ cyclic and non-cyclic alkyne species, and C₃₋₂₄ aromatic species. Any of the foregoing species can include heteroatoms such as, but not limited to, O, S, N, and P. Any of the foregoing species can also include one or more substituents selected from the group consisting of: halide groups; nitro groups; azide groups; hydroxyl groups; primary, secondary, and tertiary amine groups; aldehyde groups; ketone groups; amide groups; ether groups; ester groups; thiocyanate groups; and isothiocyanate groups.

In general, each detection moiety 224 includes one or more reporter moieties 226, each of which includes an oligonucleotide sequence. FIG. 2B is a schematic diagram showing an example of a detection moiety 224 that includes R reporter moieties 226. In general, as will be described in greater detail below, each type of capture moiety 222 is linked to a unique type of detection moiety 224 that includes a unique combination of reporter moieties 226.

Detection moiety 224 can generally include any number R of reporter moieties 226. In some embodiments, for example, R is 1 or more (e.g., 2 or more, 3 or more, 5 or more, 7 or more, 10 or more, 15 or more, 20 or more, 25 or more, 30 or more, 35 or more, 40 or more, 50 or more, or even more). Each of the reporter moieties 226 in detection moiety 224 can generally and independently have any of the properties and features described herein in connection with reporter moieties.

In some embodiments, each of the reporter moieties 226 in a detection moiety 224 is different (i.e., has a different nucleic acid sequence). Alternatively, in certain embodiments, one or more of the reporter moieties 226 in a detection moiety 224 has a sequence that is common to another one of the reporter moieties in the detection moiety. The number of reporter moieties that are the same in a detection moiety can be 2 or more (e.g., 3 or more, 4 or more, 5 or more, 6 or more, 7 or more, 8 or more).

In some embodiments, each reporter moiety 226 includes multiple nucleotide bases forming a nucleic acid sequence. The reporter moieties 226 of a detection moiety 224 can each have the same number of nucleotides, or one or more of the reporter moieties can have a different number of nucleotides. In certain embodiments, each of the reporter moieties has the same number of nucleotides. In some embodiments, each of the reporter moieties has a different number of nucleotides.

Each reporter moiety 226 includes a nucleic acid sequence. Each reporter moiety can independently include DNA bases (e.g., A, C, G, T), RNA bases (e.g., A, C, G, U), and any combination of DNA and/or RNA bases. Each reporter moiety can also include non-natural (e.g., synthetic) nucleotides, including DNA analogues and/or RNA analogues. Examples of such synthetic analogues include, but are not limited to, peptide nucleic acids (PNAs), morpholino and locked nucleic acids (LNAs), glycol nucleic acids, and threose nucleic acids.

The length of a reporter moiety (e.g., the number of nucleotides in a reporter moiety) can generally be selected as desired to ensure efficient and selective hybridization with a catalytic agent. In some embodiments, a (or each) reporter moiety can include at least 5 (e.g., at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100) nucleotides.

In certain embodiments, a (or each) reporter moiety can have between 5-30, between 5-25, between 5-20, between 10-20, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides.

In some embodiments, a (or each) reporter moiety can have no more than 5 (e.g., no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100) nucleotides.

In some embodiments, a (or each) reporter moiety can be fully single stranded. Alternatively, in certain embodiments, one or more reporter moieties can be at least partially double stranded. A partially double stranded portion of a reporter moiety can be at the 3′ end of the label region, at the 5′ end of the label region, or between the 5′ end and 3′ ends of the reporter moiety.

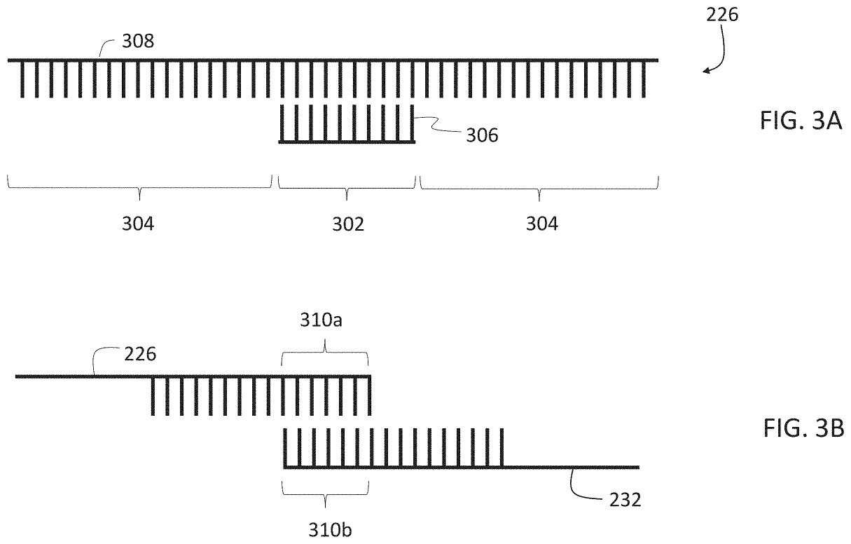

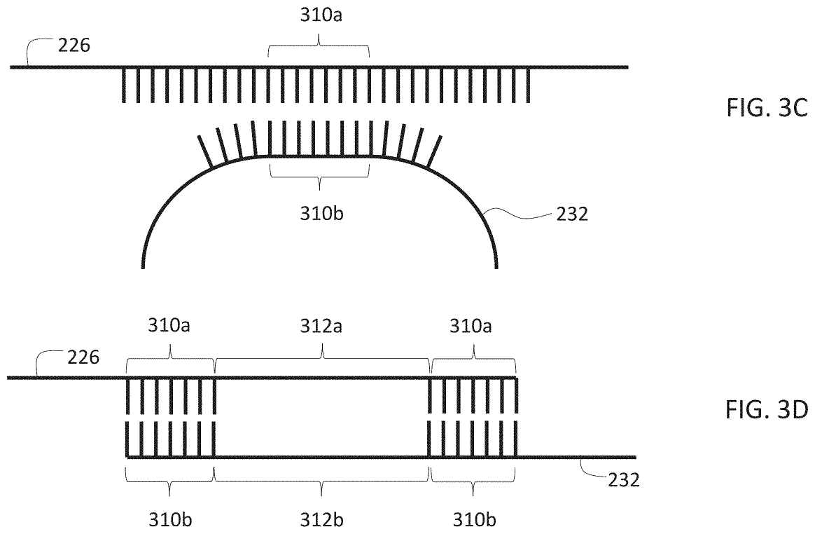

FIG. 3A is a schematic diagram of a reporter moiety 226 that includes two single stranded regions 304 and a double stranded region 302. As discussed above, double stranded region 302 can be positioned at the 3′ end of reporter moiety 226, at the 5′ end of reporter moiety 226, or at an intermediate position between the 3′ and 5′ ends. In certain embodiments, reporter moiety 226 can include more than one double stranded region (e.g., two or more, three or more, four or more, five or more, or even more double stranded regions).

The double stranded region can be formed by a secondary oligonucleotide strand 306 that is bound (e.g., hybridized) to a primary oligonucleotide strand 308 of reporter moiety 226, as shown in FIG. 3A. Alternatively, or in addition, reporter moiety 226 can include a secondary structure that allows folding of a single stranded reporter moiety 226. At least partial complementarity between different portions of the single strand allows the portions to hybridize, forming one or more double stranded regions from the single strand.

The one or more double stranded regions 302 of reporter moiety 226 can each, and collectively, extend over a percentage of the total length (e.g., the total number of nucleotides) in reporter moiety 226. In some embodiments, for example, one of more of the double stranded regions individually extends, or all of the double stranded regions collectively extend, over 1% or more (e.g., 5% or more, 10% or more, 15% or more, 20% or more, 25% or more, 30% or more, 35% or more, 40% or more, 50% or more) of the total length of reporter moiety 226.

In some embodiments, different probes 220 are bound to different RNAs 210 in a sample, and the detection moieties 224 of each probe 220 include the same number of reporter moieties 226. In certain embodiments, however, the detection moieties 224 of at least one (e.g., two or more, three or more, five or more, 10 or more, 20 or more, 30 or more, 50 or more, 100 or more) of the probes 220 include a different number of reporter moieties 226 from the detection moieties of other probes. In certain embodiments, a sample can include 2 or more (e.g., 3 or more, 4 or more, 5 or more, 6 or more, or even more) groups of probes 220, where each group of probes includes detection moieties with a different number of reporter moieties relative to the detection moieties of probes in the other groups.

As shown in FIG. 2A, probe 220 is attached to RNA 210 through capture moiety 222, which binds RNA 210 (i.e., via hybridization to all or a portion of RNA 210). In general, a hybridization region between capture moiety 222 and a portion of RNA 210 (i.e., the region of nucleotide complementarity over which capture moiety 222 hybridizes to RNA 210) can be 1 nucleotide or more (e.g., 2 nucleotides or more, 3 nucleotides or more, 4 nucleotides or more, 5 nucleotides or more, 6 nucleotides or more, 8 nucleotides or more, 10 nucleotides or more, 15 nucleotides or more, 20 nucleotides or more, 25 nucleotides or more, 30 nucleotides or more, 40 nucleotides or more, 50 nucleotides or more, 60 nucleotides or more, 70 nucleotides or more, 80 nucleotides or more, 90 nucleotides or more, 100 nucleotides or more, or even more) in length.

In some embodiments, probe 220 can optionally be coupled to the sample in which RNA 210 is located. For example, probe 220 can be bound to the sample via one or more covalent bonds. In certain embodiments, the covalent bonds can be formed by cross-linking one or more portions of probe 220 to the sample. A variety of different methods can be used to cross-link probe 220 to the sample.

For example, in some embodiments, probe 220 can include 5-(3-aminoallyl)-dUTP. Following hybridization of probe 220 to RNA 210, probe 220 can be cross-linked to the sample by introducing a bifunctional NHS ester (such as, for example, PEGylated bis(sulfosuccinimydl)suberate (BS(PEG)9)). Cross-linking can be performed any time after hybridization of probe 220 to RNA 210 occurs. Additional aspects of these methods and other cross-linking methods are described, for example, in U.S. Patent Application Publication No. US 2018/0051322, the entire contents of which are incorporated herein by reference.

A variety of other methods can also be used to cross-link or otherwise fix probe 220 in position within the sample. Such methods include, but are not limited to, linking through cell surface functional groups and DNA-binding proteins. Features and aspects of such methods are described, for example, in U.S. Pat. Nos. 9,327,036 and 10,253,333, the entire contents of each of which are incorporated by reference herein.

Returning to FIG. 1, in the next step 104, target RNA 210 is contacted with a catalytic agent that associates with one of the reporter moieties of detection moiety 224. This step is illustrated schematically in FIG. 2C. In FIG. 2C, a catalytic agent 230 contacts RNA 210. Catalytic agent 230 includes an oligonucleotide 232 conjugated to a reactive species 234. Oligonucleotide 232 is at least partially complementary to reporter moiety 226a, so that oligonucleotide 232 and reporter moiety 226a hybridize. In this manner, catalytic agent 230 localizes in the sample at the same positions as probe 220, and therefore, at the positions corresponding to RNA 210.

It should be noted that if detection moiety 224 includes more than one reporter moiety with the same oligonucleotide sequence, then catalytic agent 230 will hybridize to each of the reporter moieties with the same oligonucleotide sequence. For example, in FIG. 2C, if reporter moieties 226a and 226c have the same oligonucleotide sequence, then one molecule of catalytic agent 230 will hybridize to reporter moiety 226a, and another molecule of catalytic agent 230 will hybridize to reporter moiety 226c.

In general, oligonucleotide 232 can include any of the features described above for reporter moiety 226. Oligonucleotide 232 can, in some embodiments, include the same number of nucleotides as reporter moiety 226. Alternatively, in certain embodiments, oligonucleotide 232 can include a different number of nucleotides.

Oligonucleotide 232 can have the same or different strand structure as reporter moiety 226. That is, oligonucleotide 232 can be single stranded, double stranded, or partially double stranded, irrespective of the structure of reporter moiety 226. Oligonucleotide 232 can generally include any number of double stranded regions, as described above for reporter moiety 226, extending over a portion of the total length of oligonucleotide 232.

As discussed above, oligonucleotide 232 hybridizes to reporter moiety 226 via base pairing so that probe 220 and catalytic agent 230 are co-localized at the location of target RNA 2109. The efficiency of hybridization is related in part to the extent of complementarity between the sequences of reporter moiety 226 and oligonucleotide 232. As used herein, the percentage to which the sequences of the two sequences are complementary refers to the percentage of nucleotides in the shorter of the two sequences that have a complementary counterpart at a complementary location in the other sequence, such that the two counterparts pair during hybridization. In some embodiments, for example, the sequences of the two oligonucleotides are at least 70% (e.g., at least 75%, at least 80%, at least 85%, at least 90%, at least 95%, at least 99%) complementary.

As used herein, the term “at least partially complementary” means that two nucleotide sequences are sufficiently complementary that they hybridize. In general, two nucleotide sequences are at least partially complementary if their sequences are at least 50% complementary.

In general, oligonucleotide 232 includes at least one binding region that hybridizes to a corresponding binding region of reporter moiety 226. The binding region can be located at the 3′ end, at the 5′ end, or intermediate between the two ends, of second oligonucleotide. Where oligonucleotide 232 includes multiple binding regions, any of the binding regions can be located as above.

In some embodiments, the binding region of oligonucleotide 232 is at least partially complementary to, and hybridizes with, the 3′ end of reporter moiety 226. In certain embodiments, the binding region of oligonucleotide 232 is at least partially complementary to, and hybridizes with, the 5′ end of reporter moiety 226. FIG. 3B shows a schematic diagram of reporter moiety 226 and oligonucleotide 232 with respective binding regions 310a and 310b. Binding region 310b of oligonucleotide 232 is at least partially complementary to a 3′ or 5′ end of reporter moiety 226 in FIG. 3B.

In certain embodiments, the binding region 310b of oligonucleotide 232 is at least partially complementary to, and hybridizes with, an intermediate region of reporter moiety 226. FIG. 3C shows a schematic diagram in which binding region 310b of oligonucleotide 232 binds with an intermediate binding region 310a of reporter moiety 226.

In some embodiments, the binding region 310b of oligonucleotide 232 is at least partially complementary to, and hybridizes with, the entire reporter moiety 226. In certain embodiments, the binding region 310a of reporter moiety 226 is at least partially complementary to, and hybridizes with, the entire oligonucleotide 232.

In certain embodiments, one or both of reporter moiety 226 and oligonucleotide 232 includes multiple binding regions separated by one or more non-binding regions. FIG. 3D is a schematic diagram showing reporter moiety 226 and oligonucleotide 232, each of which includes multiple binding regions 310a and 310b, respectively, separated by non-binding regions 312a and 312b, respectively. In general, each of the binding regions can have any of the properties discussed above in connection with reporter 226 and oligonucleotide 232 and their respective binding regions.

The non-binding regions 312a and 312b can be formed by a variety of different linking species, including non-complementary nucleotide sequences and spacer moieties that do not include nucleotides. Non-binding regions 312a and 312b can have the same or different geometric lengths, and binding regions 310a and 310b can have the same or different lengths (e.g., the same or different numbers of nucleotides). Within each of reporter moiety 226 and oligonucleotide 232, binding regions and non-binding regions can have the same or different lengths.

As discussed above, oligonucleotide 232 includes a reactive species 234 conjugated to oligonucleotide 232. Reactive species 234, as will be discussed further below, reacts with a labeling agent. Reactive species 234 can correspond to any one or more of a variety of different chemical or biochemical species and moieties. In some embodiments, for example, reactive species 234 corresponds to a catalytic moiety that catalyzes a reaction of the labeling. Examples of catalytic moieties that can correspond to reactive species 234 include, but are not limited to, enzymes, transition metal-based organometallic moieties, peroxide containing moieties, and photoactivatable species. Examples of suitable enzymes include, but are not limited to, horseradish peroxidase (HRP) and soybean peroxidase. In some embodiments, reactive species 234 can include a hemin-containing complex which can mimic HRP, such as hematin. Methods for conjugating enzymes to oligonucleotides are described, for example, in van Gijlswijk et al., Cytogenet Cell Genet. 75 (4): 258-262 (1996), the entire contents of which are incorporated herein by reference.

Returning again to FIG. 1, in step 106, target RNA 210 is contacted with a labeling agent. The labeling agent reacts with the reactive species 234 of the catalytic agent 230 from step 104, depositing the labeling agent in the sample at locations in proximity to catalytic agent 230 (i.e., in proximity to target RNA 210).

This step is illustrated schematically in FIG. 2D, in which a labeling agent 240 contacts target RNA 210. As shown in FIG. 2D, labeling agent 240 reacts with reactive species 234 in a reaction represented by arrow 242. The reaction deposits labeling agent 240 or a derivative thereof in the sample at locations in proximity to the catalytic agent 230, and therefore, at locations in proximity to target RNA 210. In this manner, the deposited labeling agent 240 (or a derivative thereof) is spatially co-localized with target RNA 210.

In some embodiments, as discussed above, reactive species 234 of catalytic agent 230 is an enzyme that participates an enzyme-mediated reaction to deposit labeling agent 240 (or a derivative thereof) at locations in the sample that are proximate to target RNA 210. As an example of enzyme-mediated deposition of a labeling agent, reactive species 234 can be horseradish peroxidase (HRP) or another species that mimics the activity of HRP. HRP can be used in the methods described herein as a catalytic agent for tyramide signal amplification (TSA).

To implement TSA, labeling agent 240 includes a labeling moiety conjugated to a tyramide species. When RNA 210 is initially contacted with labeling agent 240, the tyramide species is in an inactive form. However, HRP catalyzes conversion of the tyramide species to an active form that is capable of binding with the sample in which target RNA 210 is located. Following conversion of the tyramide species to its active form, the labeling agent binds to the sample at locations proximate to where it is generated (e.g., at the location of target RNA 210). FIG. 2D illustrates schematically the deposition of labeling agent 240 (which can include an active tyramide species conjugated to a labeling moiety) at in proximity to target RNA 210.

By adjusting the amount of tyramide-containing labeling agent 240 introduced and the amount of time during which the enzyme-mediated activation process continues, the amount of labeling agent 240 deposited in the sample can be controlled. As a result, a signal that is detected and that corresponds to labeling agent 240 can be “amplified”. In the context of the present disclosure, amplification refers to the linking of more than one labeling agent to a particular reporter moiety 226. The TSA technique can be used to deposit a population of labeling agents 240 (or derivatives thereof) in a sample to generate a measurable signal corresponding to a single reporter moiety 226, thereby increasing the amplitude or intensity of the measured signal corresponding to the single reporter moiety 226 relative to a signal that would otherwise be measured from a labeling agent 240 deposited in a 1:1 ratio with respect to the single reporter moiety 226.

In general, a ratio of the number of labeling agent 240 molecules that can be deposited in the sample for each reporter moiety 226 is increased beyond 1:1 by implementing the TSA methodology described above. In some embodiments, for example, the ratio is 2:1 or more (e.g., 3:1 or more, 4:1 or more, 5:1 or more, 6:1 or more, 8:1 or more, 10:1 or more, 20:1 or more, 30:1 or more, or even more).

Amplification provides a number of important advantages. First, because measurable signals corresponding to individual reporter moieties 226 are of higher amplitude or intensity than in the absence of amplification, exposure times and measurement times can be reduced. Second, due to the increased amplitude or intensity of measurement signals, reporter moieties 226 that are present in the sample at relatively low concentrations—and whose corresponding measurement signals would otherwise be relatively weak absent amplification—can be detected with greater reliability. Third, due to the increased amplitude or intensity of measurement signals, compensation for the confounding effects of tissue autofluorescence is easier, as is the detection of the measurement signals against a background autofluorescence signal that might otherwise obscure some or all of the measurement signals.

Amplification can also be used to adjust measurement signals corresponding to different reporter moieties. For example, in samples where certain target RNAs are present at significantly smaller concentrations than other target RNAs, the amplitude or intensity of measurement signals corresponding to low-concentration reporter moieties associated with the low-concentration target RNAs can be amplified so that they more closely match the amplitudes or intensities of signals corresponding to other target RNAs in the sample that are present in higher concentrations. In this manner, the range of amplitudes or intensities of the measurement signals can be reduced, so that the dynamic range of the measurement system used to detect the measurement signals can also be smaller relative to the dynamic range that would otherwise be used to measurement signals in the absence of amplification.

When the reactive species 234 corresponds to an enzyme or other catalytic agent, the enzyme or catalytic agent can mediate the deposition of labeling agent 240 in the sample via any of a variety of different types of reactions. In some embodiments, for example (such as TSA with HRP-mediated deposition of a tyramide-conjugated labeling moiety), the reaction which is mediated by the enzyme or catalytic agent is an oxidation-reduction reaction. Other examples of suitable enzyme or catalytic agent-mediated reactions include, but are not limited to, deprotonations, eliminations, radical generation reactions, deprotections, and rearrangements.

For oxidation-reduction reactions (such as TSA with HRP-mediated deposition of labeling agent 240), a variety of different oxidation and/or reduction agents can be used. In some embodiments, for example, the oxidation agent is H₂O₂. A variety of other agents can also be used.

Further, it should also be noted that while in some embodiments, deposition of labeling agent 240 (or a derivative thereof) in the sample is irreversible, in certain embodiments the deposition of labeling agent 240 in the sample is reversible, and labeling agent 240 can optionally be removed from the sample at any time following deposition by methods such as washing, one or more chemical reactions to liberate labeling agent 240, and physical methods such as heating and exposure to radiation (e.g., photocleavage or photoionization or sputtering) of the labeling agent 240.

Labeling agent 240 can be stably bound to the sample at room temperature for 48 hours or more. In some embodiments, a sample labeled with labeling agent 240 refrigerated at 4° C. can be stable for up to at least 4 weeks, and a sample frozen at −20° C. or −80° C. can remain stably bound to labeling agent 240 for up to 4 months or more. In certain embodiments, the binding is stable for at least 48 hours when sample 202 is maintained within about 5° C. of room temperature. In some embodiments, the binding is stable for at least 48 hours when the sample 202 is maintained at a temperature between 0° C. and 40° C.

Additional methods and aspects of TSA are described, for example, in Faget et al., Methods Mol. Biol. 1318: 161-72 (2015), the entire contents of which are incorporated herein by reference.

As discussed above, in some embodiments, labeling agent 240 includes one or more labeling moieties conjugated to a tyramide species or another species that reacts with reactive species 234 of catalytic agent 230. A variety of different labeling moieties can be used in labeling agent 240, depending upon the nature of the methodology used to identify and quantify target RNA 210.

In some embodiments, for example, a labeling moiety includes a dye. As used herein, a “dye” is a species that interacts with incident light, and from which emitted light can be measured and used to detect the presence of the dye in a sample. In general, a dye can be a fluorescent moiety, an absorptive moiety (e.g., a chromogenic moiety), or another type of moiety that emits light, or modifies incident light passing through or reflected from a sample where the dye is present so that the presence of the dye can be determined by measuring changes in transmitted or reflected light from the sample.

In certain embodiments, a labeling moiety can include a hapten. The hapten can subsequently (or concurrently) be bound to a dye to provide a labeling moiety that can be detected by measuring emitted, transmitted, or reflected light from the sample.

When the labeling moiety includes a dye, a wide variety of different dyes can be used. For example, the dye can be a xanthene-based dye, such as a fluorescein dye and/or a rhodamine dye. Examples of suitable fluorescein and rhodamine dyes include, but are not limited to, fluorescein isothiocyanate (FITC), 6-carboxyfluorescein (commonly known by the abbreviations FAM and F), 6-carboxy-2′, 4′, 7′, 4,7-hexachlorofluorescein (HEX), 6-carboxy-4′, 5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110.

The dye can also be a cyanine-based dye. Suitable examples of such dyes include, but are not limited to, the dyes Cy3, Cy5 and Cy7. The dye can also be a coumarin dye (e.g., umbelliferone), a benzimide dye (e.g., any of the Hoechst dyes such as Hoechst 33258), a phenanthridine dye (e.g., Texas Red), an ethidium dyes, an acridine dyes, a carbazole dye, a phenoxazine dye, a porphyrin dye, a polymethine dye (e.g., any of the BODIPY dyes), and a quinoline dye.

When the dye is a fluorescent moiety, the dye can be a moiety corresponding to any of the following non-limiting examples and/or derivatives thereof: pyrenes, coumarins, diethylaminocoumarins, FAM, fluorescein chlorotriazinyl, fluorescein, RI 10, JOE, R6G, tetramethylrhodamine, TAMRA, lissamine, napthofluorescein, Texas Red, Cy3, and Cy5.

In certain embodiments, the dye can include one or more quantum dot-based species. Quantum dot-based fluorophores are available with fluorescence emission spectra in many different spectral bands, and suitable quantum dot-based dyes can be used as labeling species in the methods described herein.

In some embodiments, a labeling moiety includes an oligonucleotide sequence. For example, the oligonucleotide sequence can be the same oligonucleotide sequence as reporter moiety 226. Alternatively, the oligonucleotide sequence can differ from the oligonucleotide sequence of reporter moiety 226. Where the oligonucleotide sequence of the labeling moiety differs from the oligonucleotide sequence of reporter moiety 226, the oligonucleotide sequence of the labeling moiety can, in certain embodiments, be uniquely associated with reporter moiety 226. In other words, the oligonucleotide sequence of labeling moiety can be unique among oligonucleotide sequences of other labeling moieties deposited in the sample, such that the labeling moiety oligonucleotide sequence is only deposited in the sample at locations corresponding to that reporter moiety 226, and not at other locations (including not at locations corresponding to other reporter moieties).

In general, where the labeling moiety includes an oligonucleotide sequence, the oligonucleotide sequences can have any of the properties described herein in connection with reporter moiety 226 and oligonucleotide 232.

Returning again to FIG. 1, after a labeling agent 240 corresponding to reporter moiety 226 has been deposited in the sample in proximity to target RNA 210, the workflow either continues or terminates depending upon whether a labeling agent 240 corresponding to each of the different reporter moieties 226 in detection moiety 224 has been deposited in the sample. For example, referring to FIG. 2C, the workflow continues until labeling agents corresponding to each of reporter moieties 226a-226d (or more generally, each of the R reporter moieties) have been deposited in the sample.

In FIG. 1, at decision step 108, if labeling agents corresponding to each of the R reporter moieties of detection moiety 224 have been deposited in the sample, the workflow ends at step 110. If not, the procedure returns to step 104, where a different catalytic agent 230 is introduced (and which associates with one of reporter moieties 226b, 226c, or 226d in FIG. 2C, for example).

Referring again to FIG. 1, after a labeling agent 240 corresponding to one of the reporter moieties 226 has been deposited in the sample in step 106, and prior to returning to step 104 to deposit a labeling agent corresponding to another one of the reporter moieties, catalytic agent 230 can be removed from the sample. In particular, because reporter moiety 226 and oligonucleotide 232 are hybridized, removal of catalytic agent 230 involves de-hybridizing reporter moiety 226 and oligonucleotide 232

De-hybridization can also be used to control the amount of labeling agent 240 that is deposited in the sample (i.e., during amplification). More particularly, de-hybridization of reporter moiety 226 and oligonucleotide 232 can be used to terminate the reaction (e.g., a catalytic reaction such as enzyme-mediated deposition of labeling agent 240) between reactive species 234 and labeling agent 240, thereby controlling the amount of time during which deposition of labeling agent 240 in the sample occurs.

Various methods can be used to achieve de-hybridization of reporter moiety 226 and oligonucleotide 232. In some embodiments, for example, de-hybridization can be achieved by exposing the sample to one or more chaotropic reagents, such as dimethyl sulfoxide (DMSO) and formamide, wherein the molar concentration of the chaotropic reagent in a solution thereof is 60% or more (e.g., 70% or more, 80% or more, 90% or more). In effect, the double-stranded oligonucleotide region formed by reporter moiety 226 and oligonucleotide 232 has a lower melting temperature than the double-stranded region formed by capture moiety 222 and target RNA 210, which allows reporter moiety 226 to be selectively dehybridized without dehybridizing capture moiety 222.

Alternatively, de-hybridization of reporter moiety 226 can be performed by toehold mediated branch migration, by TCEP reduction of incorporated disulfide moieties, by washing the sample, by heating the sample, and by combinations of the foregoing techniques.

De-hybridization of reporter moiety 226 and oligonucleotide 232, followed by a washing step to remove free catalytic agent 230 following de-hybridization, yields a sample in which probe 220 remains bound to target RNA 210, and labeling agent 240 remains bound to the sample in proximity to target RNA 210. In effect, de-hybridization returns the sample to a state similar to that shown in FIG. 2A, with the added presence of labeling agent 240.

Following the end of the procedure shown in FIG. 1, the sample contains, in proximity to target RNA 210, populations of labeling agents corresponding to each of the R reporter moieties that are present in probe 220. FIG. 2E is a schematic diagram of an example showing populations of labeling agents deposited for each of the reporter moieties shown in FIG. 2D. In particular, labeling agents 240a correspond to reporter moiety 226a, labeling agents 240b correspond to reporter moiety 226b, labeling agents 240c correspond to reporter moiety 226c, and labeling agents 240d correspond to reporter moiety 226d.

Where each of the reporter moieties 226a-226d are different (e.g., have different oligonucleotide sequences) in FIG. 2E, the population of labeling agents that correspond to each reporter moiety will differ from the populations of labeling agents that correspond to the other reporter moieties. For example, each population of labeling agents may contain a different dye, or a different oligonucleotide sequence. If two or more of the reporter moieties are the same, the corresponding populations of labeling agents may also be the same. For example, if reporter moieties 226a and 226c are the same, then labeling agents 240a and 240c may also be the same.

### Branched and Multiplicative Probes and Catalytic Agents

In general, probe 220 can be implemented in a variety of ways. In some embodiments, as shown for example in FIG. 2A, probe 220 includes a capture moiety 222 and a contiguous detection moiety 224 linked to one end of capture moiety 222. In certain embodiments, detection moiety 224 is not contiguous; that is, certain portions of detection moiety 224 are linked to one position on capture moiety 222, and other portions of detection moiety 224 are linked to another position on capture moiety 222. As an example, for a detection moiety 224 that includes R reporter moieties 226, some of the reporter moieties can be linked to one end of capture moiety 222, and some of the reporter moieties can be linked to the other end of capture moiety 222.

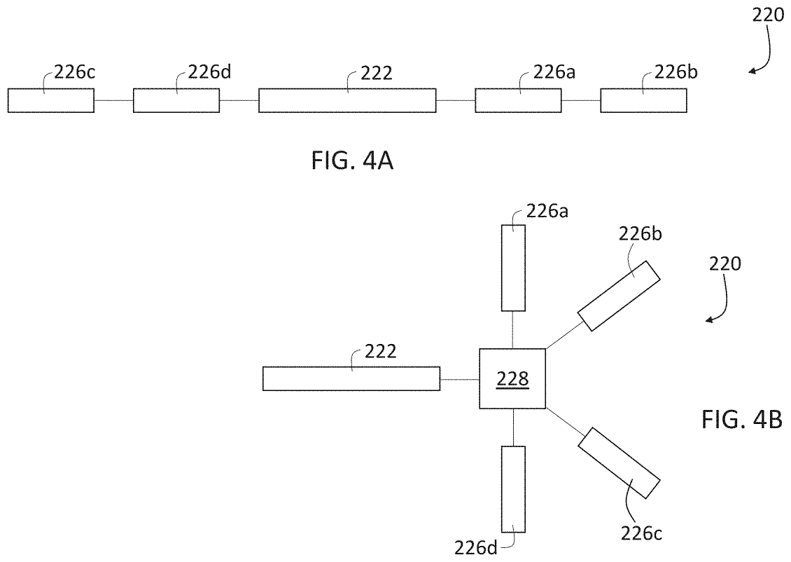

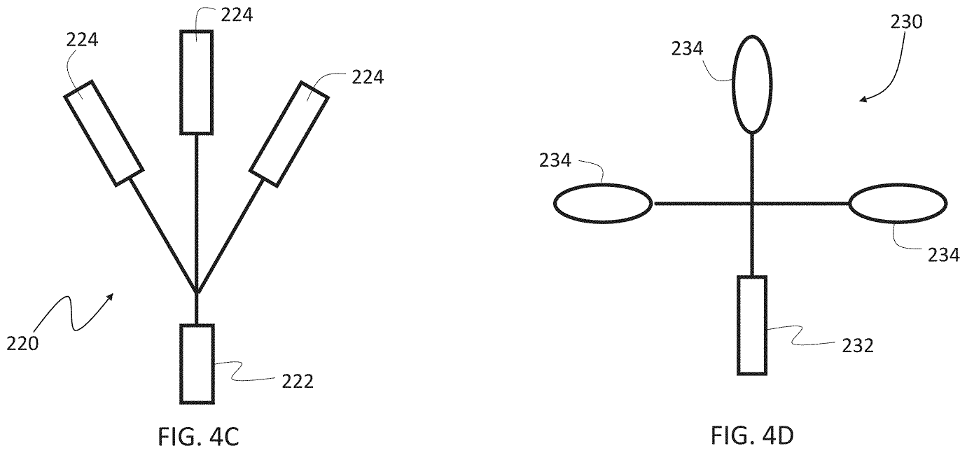

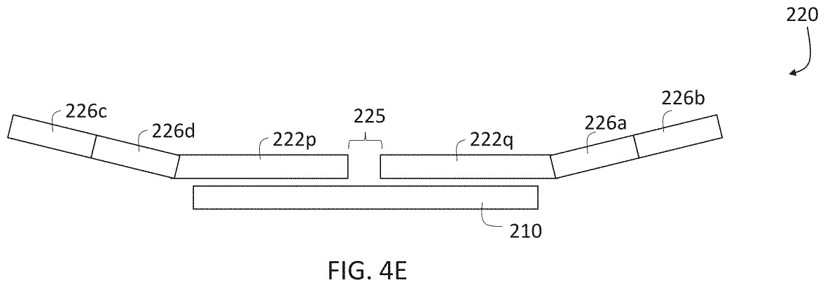

FIG. 4A is a schematic diagram showing an example of a probe 220 that includes a capture moiety 222 and four reporter moieties 226a-226d that form detection moiety 224. Reporter moieties 226a and 226b are linked to one end of capture moiety 222 and reporter moieties 226c and 226d are linked to the other end of capture moiety 222. It should be appreciated that FIG. 4A is merely an example, and the reporter moieties of detection moiety 224 can generally be distributed in any manner among different linking points on capture moiety 222.

In some embodiments, one or more of the reporter moieties of detection moiety 224 can be linked through an intermediate moiety to one another and/or to capture moiety 222. FIG. 4B shows an example of a probe 220 in which capture moiety 222 is linked to an intermediate moiety 228, and each of reporter moieties 226a-226d is also linked to intermediate moiety 228. Intermediate moiety 228 can generally be implemented as any chemical species, particle, group, or moiety including, but not limited to, nanoparticles, polymers and oligomers, and any of the linking species described herein. In certain embodiments, probe 220 can include more than one detection moiety.

Probes with multiple detection moieties can be further used to amplify signals corresponding to particular target RNAs. By linking more than one detection moiety to capture moiety 222, additional catalytic agents can be localized in proximity to target RNA 210, increasing the rate and amount of labeling agents that can be deposited in the sample in proximity to target RNA 210.

A wide variety of branched, dendritic, network, and other structures can be implemented in such probes. FIG. 4C shows an example of a probe 220 that includes a capture moiety 222 conjugated to three detection moieties 224. In general, each of the detection moieties 224 has the same set of reporter moieties 226, so that capture agents introduced can hybridize in a similar manner with the reporter moieties of each of the detection moieties 224. While three detection moieties 224 are linked to capture moiety 222 in FIG. 4C, more generally 2 or more (e.g., 3 or more, 4 or more, 5 or more, 6 or more, 7 or more, 8 or more, or even more) detection moieties 224 can be linked to capture moiety 222.

In some embodiments, catalytic agent 230 includes multiple reactive species 234. FIG. 4D is a schematic diagram showing a catalytic agent 230 in which oligonucleotide 232 is conjugated to three reactive species 234. The three reactive species can all be the same, or one or more can be different from the others. While three reactive species 234 are conjugated to oligonucleotide 232 in FIG. 4D, more generally 2 or more (e.g., 3 or more, 4 or more, 5 or more, 6 or more, 7 or more, 8 or more, or even more) reactive species can be conjugated to oligonucleotide 232. By conjugating more than one reactive species to oligonucleotide 232, additional reactive species can be localized in proximity to target RNA 210, increasing the rate and amount of labeling agents that can be deposited in proximity to target RNA 210.

### Multiplexed Deposition of Labeling Agents

In the foregoing discussion, labeling agents 240 corresponding to different reporter moieties 226 are deposited serially in a sample in proximity to target RNA 210. In some embodiments, however, labeling agents 240 can be deposited in parallel. To deposit labeling agents associated with different reporter moieties in parallel, in general, pairs of catalytic agents 230 and labeling agents 240 that react selectively with one another are used. For example, two different catalytic agents—each of which hybridizes to a different reporter moiety 226—can be contacted with a target RNA 210, selectively hybridizing the catalytic agents to their corresponding reporter moieties. Different labeling agents 240 associated with the different reporter moieties 226 are then introduced. To ensure that cross-reactivity between the catalytic agents and labeling agents is relatively small or non-existent, the catalytic agents can include different reactive species 234 (e.g., different enzymes), and the labeling agents can similarly include moieties that selectively react with only one of the reactive species 234 (e.g., different enzyme substrates).

In general, by employing pairs of specific and non-interfering catalytic agents and labeling agents, two or more (e.g., three or more, four or more, five or more, six or more, seven or more, eight or more, ten or more, 12 or more, 15 or more, or even more) labeling agents can be deposited and correctly localized in a sample in parallel.

### Multiplexed Analysis of Target RNAs

While the foregoing discussion focuses on the deposition of populations of labeling agents in proximity to a single type of target RNA 210 in sample, more generally the methods described herein can be used to deposit populations of labeling agents in proximity to multiple different types of target RNAs in a sample. The populations of labeling agents for the different types of target RNAs can be deposited serially or in parallel.

To deposit populations of labeling agents for each different type of target RNA in series, the procedure shown in FIG. 1 can be repeated for each different type of target RNA in a sample, with a different probe 220 (having a different capture moiety 222) used to selectively bind each different type of target RNA.

To deposit populations of labeling agents for different types of target RNAs in parallel, a sample containing the different types of target RNAs is exposed to multiple pluralities of probes, where each plurality of probes includes a different capture moiety 222 that selectively binds to a different RNA or to a different portion of the same RNA in the sample. Each plurality of probes represents a different type of probe that selectively binds to a different type of RNA or to a different portion of the same RNA in the sample.

To implement such a workflow, a composition is prepared that contains populations of different types of probes. For each probe type, the members of the corresponding population of probes each have a common capture moiety 222 that selectively binds to a corresponding type of RNA 210 and/or to a corresponding portion of a type of RNA 210 (i.e., a RNA with a specific oligonucleotide sequence), and a detection moiety 224 that includes a common set of reporter moieties 226. In some embodiments, the set of reporter moieties is the same among all detection moieties of the probe type. In certain embodiments, all detection moieties of the probe type contain the same set of reporter moieties, and certain detection moieties can also include additional reporter moieties and/or other structural features.

In some embodiments, the composition includes more than one probe type that binds to the same type of RNA. For example, the composition can include a first probe type that binds to a first portion of a type of RNA (i.e., a RNA transcript having a particular oligonucleotide sequence), and a second probe type that binds to a second portion of the same type of RNA. Detection moieties of the first and second probe types can be the same or different.

More generally, for a particular type of RNA, the composition can include one or more (e.g., two or more, three or more, four or more, five or more, six or more, seven or more, eight or more, nine or more, ten or more, or even more) different types of probes, each of which binds to a different portion (e.g., a different oligonucleotide sequence) of the type of RNA. Each of the different types of probes that binds to the same type of RNA can have a different type of capture moiety 222 (i.e., a capture moiety 222 that binds to a different portion of the oligonucleotide sequence of RNA 210). Among the different types of the probes, the capture moieties can have the same or different numbers of nucleotides, and the capture moieties can individually have any of the properties discussed herein in connection with capture moieties.

In some embodiments, among the different types of probes that bind different portions of the same type of RNA, the detection moieties 224 are the same. That is, the composition includes two or more different types of probes, each of which binds to a different portion of the same type of RNA 210, and each of which includes the same detection moieties (i.e., the same set of reporter moieties 226 in the detection moieties 224). Alternatively, the composition can include two or more different types of probes, each of which binds to a different portion of the same type of RNA 210, and each of which includes detection moieties with the same type of reporter moieties 226, but the number of reporter moieties 226 in some or all of the different types of detection moieties 224 can be different.

In certain embodiments, one or more of the detection moieties among the different types of probes are different from the other detection moieties of the different probes. For example, in some embodiments, detection moieties differ according to the number of reporter moieties 226 in the detection moieties. In certain embodiments, detection moieties differ according to the type of reporter moieties 226 present in the detection moieties. In some embodiments, detection moieties differ according to both the number and type of reporter moieties 226 present in the detection moieties. In general, the reporter moieties 226 in the detection moieties can individually have any of the properties of reporter moieties discussed herein.

Exposure of the sample to pluralities of different types of probes yields a sample in which different types of RNAs are labeled with different types of probes, where each different type of probe has a different type of detection moiety 224 with a set of reporter moieties that is unique a distinguishable from the sets of reporter moieties that are part of the detection moieties of other types of probes. In this manner, each unique set of reporter moieties is selectively associated with only one type of target RNA to which the unique corresponding probe (which comprises the unique set of reporter moieties) binds. Samples in which different types of RNAs have been selectively labeled with different probes can then be further treated, as discussed above, to deposit unique sets of populations of different labeling agents in proximity to each of the different types of RNAs.

### Multi-Cycle Detection of Labeled RNAs

With one or more types of RNAs 210 in a sample labeled with different sets of labeling agents, the RNAs in the sample can be detected and quantified through successive cycles of optical imaging. In general, the analytical procedure differs depending upon the nature of the labeling agents that are deposited in the sample. For a sample in which target RNAs are labeled with labeling agents that include a dye labeling moiety, images of the sample can be obtained directly, with measured signals corresponding to the various dye labeling moieties.

Alternatively, for a sample in which target RNAs are labeled with labeling agents that include oligonucleotide sequences that are the same as, or correspond uniquely to, the oligonucleotide sequences of the reporter moieties, an additional step is performed. The sample is contacted with optical labels, each of which consists of an oligonucleotide sequence that is complementary to, and hybridizes with, one of the oligonucleotide sequences of the labeling agents, linked to a species that generates an optical signal. The species that generates the optical signal can correspond, for example, to any of the dyes described herein.

By measuring the optical signals generated by each dye labeling moiety or optical label, individual RNAs in the sample can be identified and quantified, as each different type of RNA in the sample is associated with a different combination of optical signals arising from the labeling moieties (and, optionally, the optical labels that hybridize to the labeling moieties). As such, the combination of measured optical signals associated with each type of RNA is unique.

The oligonucleotides of the optical labels each have a sequence that is at least partially, or fully, complementary to one of the oligonucleotides of labeling agents 240 deposited in the sample. Oligonucleotides of the optical labels can each have the same or a different number of nucleotides. More generally, the oligonucleotides of the optical labels can have the same properties as the other types of oligonucleotides discussed above.

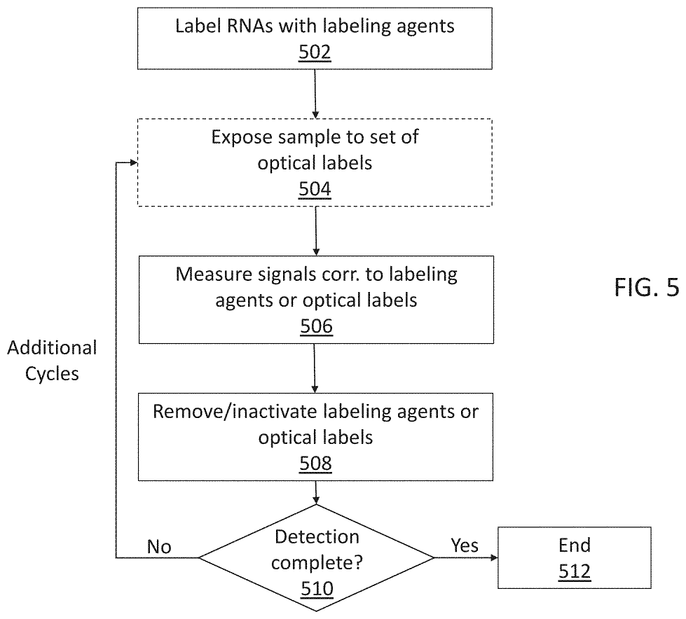

To detect RNAs in a sample, one or more cycles of optical imaging (which can optionally include optical label hybridization) are generally performed. FIG. 5 is a flow chart that shows a set of example steps for detecting and quantifying multiple types of RNAs in a sample. In step 502, the multiple types of RNAs are labeled with unique sets of labeling agents as described above. Deposited in the sample in proximity of each type of RNA is a unique set of populations of multiple labeling agents. While different types of RNAs may be labeled with one or more common labeling agents, each combination of different labeling agents is uniquely associated with a different type of RNA.

Next, in step 504, the sample is optionally exposed to a set of one or more optical labels. The set of optical labels typically includes between 1 and 8 different optical labels (e.g., two, three, or four different optical labels), but can generally include any number of optical labels in each cycle of the flow chart of FIG. 5. As noted above, when target RNAs in the sample are labeled with unique combinations of labeling agents that include dyes, the sample is not exposed to optical labels in step 504. However, when target RNAs in the sample are labeled with unique combinations of labeling agents that include oligonucleotides, the sample is contacted with optical labels that selectively bind to the oligonucleotides of the labeling agents to label the target RNAs with unique combinations of dyes.

If optical labels are introduced in step 504, one or more of the optical labels can hybridize with one or more of the labeling agents in the sample. To increase the efficiency with which different types of labeling agents are identified (e.g., by reducing the number of detection cycles), the set of optical labels can be selected such that, for at least one (and more generally, more than one) of the different types of labeling agents present in the sample, multiple different optical labels of the introduced set hybridize to different labeling agents, and generate optical signals. In this manner, multiple labeling agents can be identified in a single detection cycle, reducing the number of cycles required to fully elucidate all of the labeling agents present in the sample. By selecting the optical label set in each cycle such that multiple different optical labels hybridize to different labeling agents present in the sample, the number of detection cycles can be more efficiently utilized to identify the different reporter moieties, and therefore, to detect specific RNAs in the sample.

Conversely, as a simplification of the above general optical label selection and utilization methodology, in some embodiments, the set of optical labels can optionally be selected such that in step 504, at most one of the optical labels in the set hybridizes to one member of the set of labeling agents associated with each type of RNA. In other words, in each detection cycle that includes step 504, at most one of the reporter moieties associated with each different types of RNA can be identified. As a consequence of this constraint on the selection of optical labels, each type of RNA that is associated with D different reporter moieties (through a corresponding probe 220) can be fully identified following a minimum of D detection cycles. As the number of different types of RNAs increases, the number of detection cycles that are performed to fully identify each of the different types of reporter moieties generally increases, particularly if the number of different optical labels in each set remains constant.

While this choice of the optical label set may be less efficient than the more general methodology in which more than one optical label hybridizes to a set of labeling agents associated with a particular type of RNA, this choice of optical label set can be useful in some circumstances. For example, in samples where optical labels may interact with one another (e.g., by fluorescence resonance excitation transfer, donor-acceptor quenching, or photoinduced electron transfer), it may be disadvantageous to introduce certain optical labels in a manner such that they hybridize to a common labeling agent, as positioning the optical labels in such close proximity may promote such interactions, which disrupt successful detection of the label regions to which they hybridize by extinguishing, masking, or otherwise interfering with the signals generated by the optical labels. In such circumstances, by selecting the set of optical labels so that at most one optical label from the set hybridizes to each set of labeling agents associated with a particular type of RNA in the sample, interactions between different types of optical labels can be reduced.

Next, in step 506, optical signals corresponding to the labeling agents (if the labeling agents include dyes) or optical labels (if optical labels that include dyes are introduced) are measured. In some embodiments, the optical signals are measured by obtaining one or more images (e.g., multispectral images) of the sample with the labeling agents in the sample (or the optical labels hybridized to complementary labeling agents in the sample). To obtain the one or more images, the sample is exposed to incident light, and signal radiation generated by the labeling agents or optical labels (e.g., fluorescence emission) is detected using an imaging detector such as a CCD array or CMOS-based array detector.

In general, each of the different labeling agents or optical labels in the sample generates signal radiation according to a different spectral distribution, and is therefore associated with a different detection channel. In practice, signal radiation in different detection channels can be detected in a variety of ways. In some embodiments, where each detection channel is well separated spectrally from the other detection channels, the signal radiation generated by each different type of labeling agent or optical label is relatively well isolated spectrally in a distinct detection channel. As such, signal radiation attributable to each of the different types of labeling agents or optical labels can readily be isolated and detected by spectral filtering (e.g., with a plurality of optical bandpass filters) and/or by using a spectrally resolving detector, such as a grating, prism, or other spectrally dispersive element in conjunction with a CCD array or CMOS-based array detector.

In certain embodiments, the spectral distributions of signal radiation generated by the different labeling agents or optical labels may overlap to a degree that is not insignificant, such that optical filtering and spectral dispersion methods alone are insufficient to isolate signal radiation generated by each of the different labeling agents or optical labels. Because the spectral distributions of the signal radiation are spectrally convolved to some extent, accurate detection of signals generated by each of the labeling agents or optical labels may therefore involve more complex spectral deconvolution techniques to accurately separate and assign measured signals to specific labeling agents or optical labels.

In such circumstances, sample images that include signal radiation from multiple different labeling agents or optical labels can optionally be decomposed into a set of images, in which each image in the set corresponds substantially only to signal radiation from one optical label. A variety of methods can be used to perform such decompositions, including for example spectral unmixing methods that involve performing an eigenvector decomposition of the measured optical signals into individual contributions from “pure” spectral components (e.g., contributions from each optical label).

In general, RNAs are present in the sample at different spatial locations. As such, for each pixel in an image of the sample, any optical signals measured at that pixel will arise from labeling agents or optical labels associated with a particular detection moiety linked to a capture moiety that specifically binds to one RNA at a location in the sample corresponding to the pixel. In embodiments where, in a detection cycle, multiple labeling agents or optical labels associated with a single RNA are present and detected, multiple optical signals corresponding to the single are detected at certain pixels. As described above, if the multiple optical signals are well separated spectrally, they can be resolved by spectral filtering methods. Alternatively, if the multiple optical signals overlap spectrally, the total measured signal at the pixel—which corresponds to a convolution of signals due to multiple labeling agents or optical labels—can be decomposed into component signals (e.g., component images) that individually correspond to an optical signal generated substantially only by a single one of the labeling agents or optical labels.

Further, as described above, if the set of optical labels is selected so that, at most, one optical label in the set hybridizes to the labeling moieties associated with each RNA in the sample, then each pixel in the set of one or more images will include, at most, an optical signal corresponding to one of the optical labels in the set. In such circumstances, methods for spectrally decomposing the measured signals are generally not required to separate signals generated by multiple optical labels. However, such methods can still be used, for example, to compensate for the effects of autofluorescence and spectral contributions that may arise from other species present in the same that are not of interest.

It should also be noted that in each image of the sample, certain pixels may be “dark” (i.e., have zero or very low aggregate signal intensity). A dark pixel indicates that none of the optical labels hybridized to reporter moieties at a location in the sample corresponding to the dark pixel during the current detection cycle. If no optical signals are measured at a particular pixel among all images through all detection cycles, then none of the different types of target RNAs was present at the location in the sample corresponding to the dark pixel.

Step 506 yields a set of one or more images of the sample. Particular pixels at a common location in the set of images correspond to the same location in the sample, which is represented by the common pixel location in the images. Collectively, pixels across the set of images that correspond to a common pixel location are associated with optical signals generated by labeling agents or optical labels at the corresponding location in the sample. Because the optical signals generated by each different type of labeling agent or optical label in a detection cycle are known, the current or former presence of particular reporter moieties (see step 102 of FIG. 1) of probe 220 at each pixel location in the images can be determined. After all detection cycles are complete, the particular detection moiety consisting of a set of reporter moieties present at each pixel location can be used to determine the type of probe 220 that binds at the pixel location, and therefore, the RNA present at the pixel location.

Following step 506, the set of labeling agents or optical labels can be inactivated or removed from the sample in step 508. A variety of different methods can be used in step 508 for removal or inactivation.

In some embodiments, optical labels that hybridize to deposited labeling agents can be readily removed by dehybridization. Dehybridization can be accomplished using various methods including, but not limited to: exposure to one or more chaotropic reagents; thermally-induced dehybridization via heating; toehold mediated strand displacement (TMSD); and enzymatic strand displacement using enzymes such as RNAse, DNAse.

In certain embodiments, portions of hybridized optical labels can be removed from the sample, while other portions of the optical labels remain in the sample. For example, one or more reducing agents can be used to cleave covalent bonds that link a dye moiety to an oligonucleotide in an optical label. The cleaved dye moieties can then be washed from the sample. The oligonucleotides can optionally remain hybridized to deposited labeling agents in the sample. A variety of different reducing agents can be used for this purpose. For example, tri(2-carboxyethyl)phosphine (TCEP) can be used to cleave dye moieties that are linked via disulfide bonds to oligonucleotides in optical labels.

In some embodiments, optical labels are not removed from the sample, but are instead inactivated so that they do not generate optical signals in subsequent detection cycles. Labeling agents deposited in the sample that contain dyes can also be inactivated in this manner following imaging. Various methods can be used for inactivation of labeling agents or optical labels. For example, in certain embodiments, chemical bleaching can be used to inactivate the labeling agents or optical labels.

Next, in step 510, if analysis of the RNAs present in the sample is complete, then the workflow ends. However, if analysis is not complete, one or more additional cycles of steps 504 (optional), 506, and 508 are performed. In each additional cycle, a set of optical labels is optionally introduced into the sample, optical signals corresponding to the labeling agents or optical labels are measured and optionally decomposed as described above, and the labeling agents or optical labels can optionally be inactivated or removed from the sample.

The workflow shown in FIG. 5 can be repeated for any number of cycles to detect and quantify RNAs in the sample. In some embodiments, where optical labels are introduced (e.g., step 504) and hybridize to labeling agents deposited in the sample, each set of optical labels that is used in a particular cycle is unique; that is, none of the optical labels are re-used in subsequent cycles. In this manner, labeling agents deposited in the sample that are associated with a particular type of reporter moiety are detected only once through the entire workflow.

In some embodiments, step 502 of the workflow shown in FIG. 5 is performed only once. That is, all RNAs of interest in the sample are initially labeled with labeling agents, and then pools consisting of one or more optical labels are introduced in one or more cycles to measure signals corresponding to the labeled RNAs. In certain embodiments, step 502 of the workflow shown in FIG. 5 is performed more than once (in effect, the entire workflow of FIG. 5 is performed more than once). That is, in step 502, a first subset of all RNAs of interest in the sample are labeled with labeling agents, and the labeled RNAs are exposed to pools of one or more optical labels in each pool, in one or more cycles of steps 504-508, to detect the first subset of RNAs. Then, the a second subset of all RNAs of interest in the sample are labeled with labeling agents by repeating step 502, and the labeled RNAs of the second subset are exposed to pools of one or more optical labels in each pool, in one or more cycles of steps 504-508, to detect the second subset of RNAs. This procedure can be performed with any number (e.g., two, three, four, five, six, eight, ten, or even more) subsets of all of the RNAs of interest in the sample, which may effectively amount to performing the workflow of FIG. 5 any number of times until RNAs of interest in the sample have been detected.

In certain embodiments, to perform error checking and/or to correct for binding and imaging errors such as incomplete hybridization and/or optical imaging aberrations, certain labeling agents or optical labels can be imaged in more than one cycle in FIG. 5. By doing so, for example, optical signals measured in prior cycles can be verified, and optical signals that may have been partially absent in prior cycles (e.g., due to incomplete hybridization of optical labels or processes such as radiation transfer or quenching among labeling agents or optical labels) can be measured in a later cycle.

It should also be noted that while in some embodiments, where optical labels that hybridized to labeling agents deposited in the sample are introduced into the sample only once during the workflow shown in FIG. 5, optical labels that bind to different labeling agents, but have the same dye, can be used in different cycles of the workflow. That is, provided that different optical labels in the same cycle do not include the same dye, measured signals can be unambiguously attributed to the different optical labels. This unambiguous assignment remains possible when different optical labels include the same dye, provided the different optical labels are applied—and corresponding signals measured—during different cycles in FIG. 5.

In general, the workflow in FIG. 5 typically continues until a sufficient number of cycles have been performed to unambiguously identify each of the target RNAs in the sample. In some embodiments, the workflow continues until signals attributable to labeling agents or optical labels associated with each of the reporter moieties of each of the different types of probes have been measured.

In certain embodiments, depending upon the number of target RNAs, the number of different detection moieties, and the number of reporter moieties in each detection moiety, it may be possible to unambiguously identify each of the target RNAs without measuring optical signals arising from labeling agents or optical labels associated with every reporter moiety of every detection moiety. For example, probes targeting RNAs in the sample can include detection moieties with 6 reporter moieties, but it may be possible to unambiguously identify certain target RNAs of interest by measuring optical signals from labeling agents or optical labels associated with only a subset (e.g., 3 or 4) of the reporter moieties of some or all of the detection moieties, if the distributions of reporter moieties among the detection moieties are sufficiently differentiated.

In this manner, one or more detection cycles may be omitted, saving the expense and time associated with the omitted cycles. This method may be applied, for example, to assays in which a limited number of target RNAs are of interest, while the generalized assay permits identification of a much larger number of different RNAs. Foregoing detection cycles that only provide information about RNAs that are not of interest, or that do not provide additional information that is useful for identifying RNAs that are of interest, shortens the time associated with the assay, and may reduce the associated cost by limiting the number of different optical labels that are used.

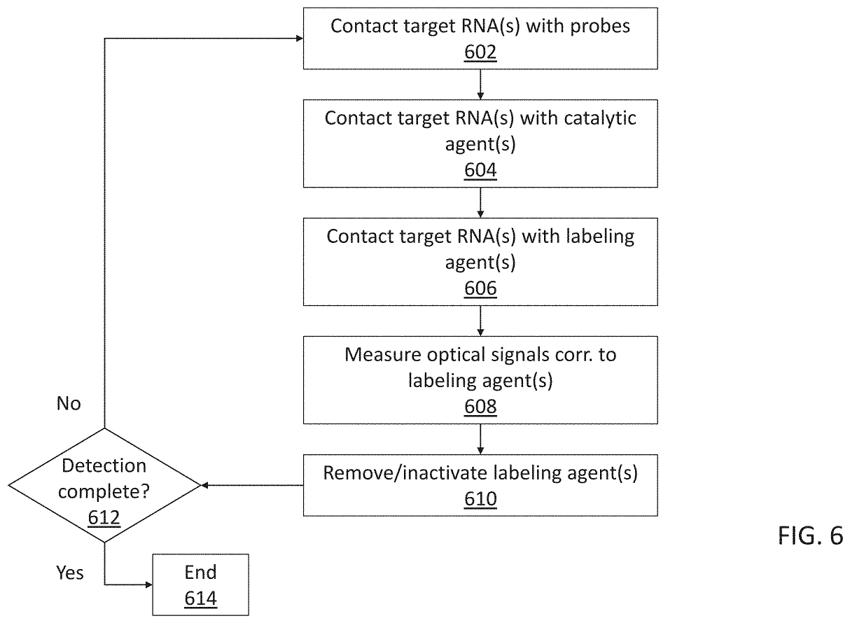

An optional workflow that is suited in particular to the deposition and imaging of labeling agents with dyes (i.e., without applying optical labels) is shown in FIG. 6. In the workflow of FIG. 6 labeling agents are deposited in the sample and imaged in the same cycle of the workflow. Accordingly, an important consideration is to ensure that within each cycle of the workflow, the dyes that are deposited in the sample are unambiguously attributable (e.g., each dye corresponds to only one type of reporter moiety), and further, that signals corresponding to the dyes remain spectrally distinguishable. Consequently, the number of different types of labeling agents (each with a different dye) that can be deposited in a single cycle may be limited by these practical considerations.

In step 602, one or more target RNAs in the sample are contacted with one or more probes in a manner similar to step 102 of FIG. 1. Then, in step 604, one or more target RNAs in the sample are contacted with one or more catalytic agents, where different catalytic agents hybridized to different reporter moieties of the probes, as in step 104 of FIG. 1. Next, in step 606, the target RNAs are contacted with one or more labeling agents, which react with the catalytic agents and are deposited in the sample in proximity to the corresponding reporter agents, as described in step 106 of FIG. 1.

Following step 606, the sample contains deposited populations of one or more labeling agents, with each different type of labeling agent containing a different type of dye. The number of different types of labeling agents can generally be selected as desired, provided that, as discussed above, optical signals corresponding to the different dyes can be distinguished from one another. In some embodiments, for example, the number of different types of labeling agents can be 1 or more (e.g., 2 or more, 3 or more, 4 or more, 5 or more, 6 or more, 7 or more, 8 or more, 10 or more, 12 or more, 15 or more, 20 or more, or even more).

The probes, catalytic agents, and labeling agents applied in individual cycles of the workflow of FIG. 6 can also be selected to partially or fully identify one or more different types of target RNA. For example, in some embodiments, each cycle of the workflow shown in FIG. 6 targets only one type of target RNA. The probe applied in step 602 is one type of probe that specifically binds to only the targeted RNA. Alternatively, in certain embodiments, one or more cycles of the workflow shown in FIG. 6 target more than one type of target RNA, and the probes applied in step 602 include multiple types of probes, where each type specifically binds to a different targeted RNA.

In some embodiments, a single type of catalytic agent contacts the sample in step 604 and a single type of labeling agent contacts the sample in step 606, depositing a population of a single type of labeling agent in the sample for imaging in later steps of the workflow. Alternatively, in certain embodiments, multiple types of catalytic agents are introduced into the sample, and multiple different types of labeling agents (corresponding to different types of reporter moieties) are introduced and deposited following reaction with corresponding catalytic agents. Different types of labeling agents can be deposited in parallel, serially, or both serially and in parallel.

To deposit labeling agents in parallel, the sample is contacted with different types of catalytic agents (that selectively hybridize to different reporter moieties) at the same time. As discussed above, the different types of catalytic agents can include different types of reactive species 234, each of which selectively reacts with only one type of labeling agents, such as different types of enzymes. Then, different types of labeling agents are introduced. Selective reactions between the different types of catalytic agents and labeling agents ensures that different labeling agents are selectively deposited in the sample in proximity to different reporter moieties.

To deposit labeling agents serially, the sample is contacted with a single type of catalytic agent which selectively hybridizes to one type of reporter moiety, and then a single type of labeling agent is introduced, which reacts with the catalytic agent to deposit a population of the labeling agent in the sample in proximity to the reporter moiety. The catalytic agent is removed from the sample (e.g., via dehybridization), and then the process is repeated one or more additional times, with a different catalytic agent that selectively hybridizes to a different reporter moiety each time, and a different labeling agent that reacts with the different catalytic agent each time, followed by removal of the catalytic agent. In this manner, populations of different labeling agents are deposited in the sample, each in proximity to a different corresponding reporter moiety.

It should be noted that when multiple different types of labeling agents are deposited in the sample in a single cycle of the workflow in FIG. 6, the different types of labeling agents may correspond to the same or different detection moieties (i.e., the same or different probes, and therefore, the same or different RNAs). For example, in some embodiments, in one or more cycles of the workflow of FIG. 6, all of the different types of labeling agents deposited in the sample correspond to the same detection moiety, and thus, the same target RNA. Different types of labeling agents can be deposited for each of the different reporter moieties in the detection moiety, or for only some of the different reporter moieties in the detection moiety.

Alternatively, in certain embodiments, in one or more cycles of the workflow of FIG. 6, the different types of labeling agents deposited in the sample correspond to different detection moieties. That is, at least one of the different types of labeling agents is associated with a reporter moiety of one type of detection moiety (and therefore one type of target RNA), and at least one of the different types of labeling agents is associated with a reporter moiety of a different type of detection moiety (and therefore a different type of target RNA). In general, there is no limit to the number of different types of detection moieties (and target RNAs) to which the labeling agents deposited in the sample correspond, and the number of such different types of detection moieties to which the deposited labeling agents correspond can be 2 or more (e.g., 3 or more, 4 or more, 5 or more, 6 or more, 7 or more, 8 or more, 10 or more, 15 or more, 20 or more, or even more).

Following step 606, the sample contains populations of one or more different types of labeling agents deposited in proximity to one or more different types of reporter moieties. Next, in step 608, optical signals are measured that correspond to the one or more different types of labeling agents. Measurement of the optical signals occurs in a manner similar to step 506 of FIG. 5.

Then, in step 610, the one or more different types of labeling agents can optionally be inactivated or removed from the sample. Inactivation and/or removal can be performed as described above in connection with step 508 of FIG. 5.

At step 612, if desired optical signals corresponding to all reporter moieties of interest associated with all of the target RNAs in the sample have been measured, the workflow terminates at step 614. Alternatively, if optical signals corresponding to certain reporter moieties of interest have not yet been measured, the workflow returns to step 602, and additional probes, catalytic agents, and labeling agents can be introduced. The workflow shown in FIG. 6 can generally be repeated for any number of cycles until appropriate optical signals arising from labeling agents associated with each of the reporter moieties of interest have been measured, allowing each of the different target RNAs of interest to be identified in the sample.

The workflows in FIGS. 5 and 6 can be modified to include one or more standardization steps in which a standardization optical label, which generates an optical signal that is different from the optical signals generated by the labeling agents or optical labels associated with the reporter moieties, is introduced into the sample. The standardization optical label, for example, can hybridize to a particular portion of a labeling agent deposited in the sample, and can generate a standardization optical signal which is detected in the same manner discussed previously.

Variations in measured optical signals can be presumed to arise from sample inhomogeneity, variance in detection sensitivity, and other factors that may yield variations in imaging across a spatial field of view. The measured optical signals can be corrected for such variations, using the optical signal from the standardization optical label as a reference signal. A wide variety of such corrections are possible, including but not limited to dividing measured optical signals by the corresponding standardization optical label signal (i.e., a spatially-resolved correction), normalizing or scaling measured optical signals to yield signals that deviate reproducibly from the reference standardization optical signal, and using the standardization optical signal to establish thresholds for positive and negative signal detection among the measured optical signals.

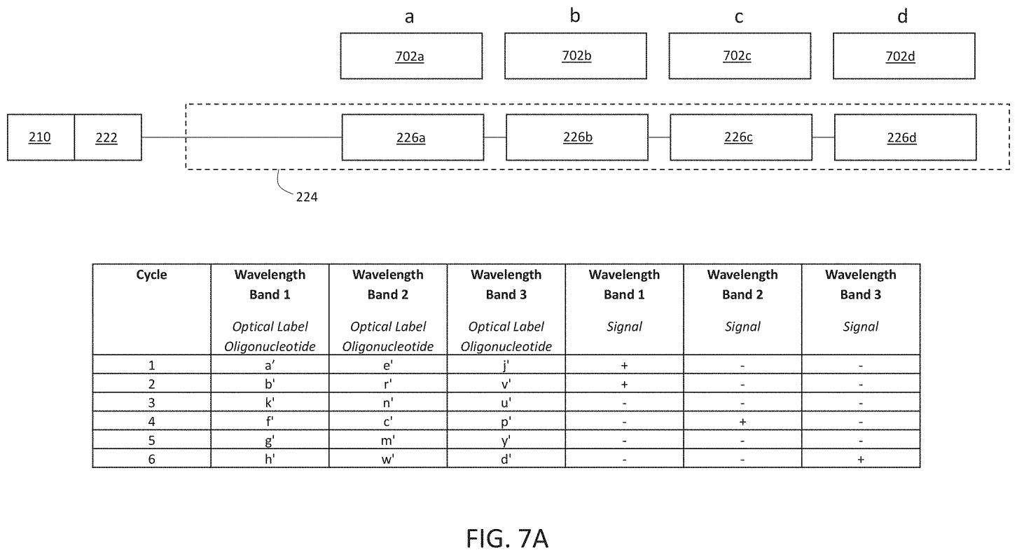

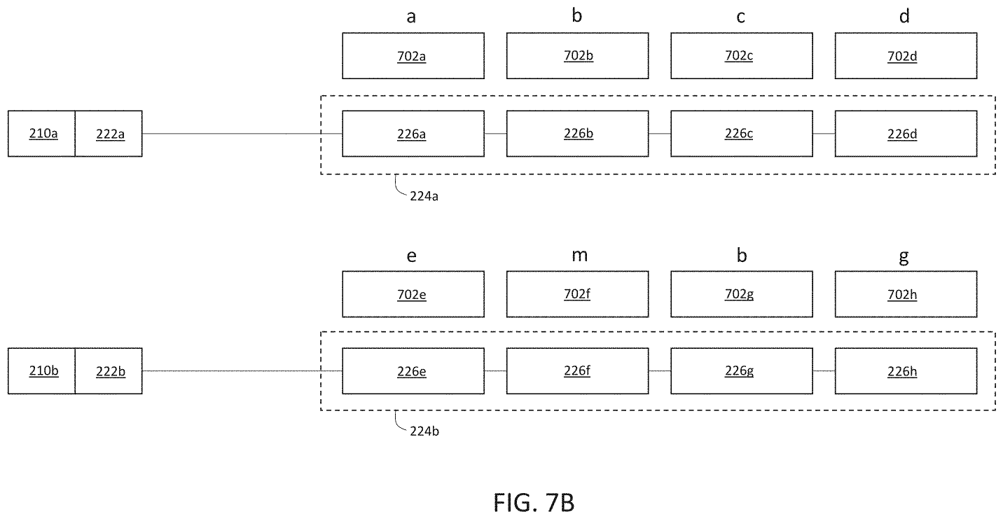

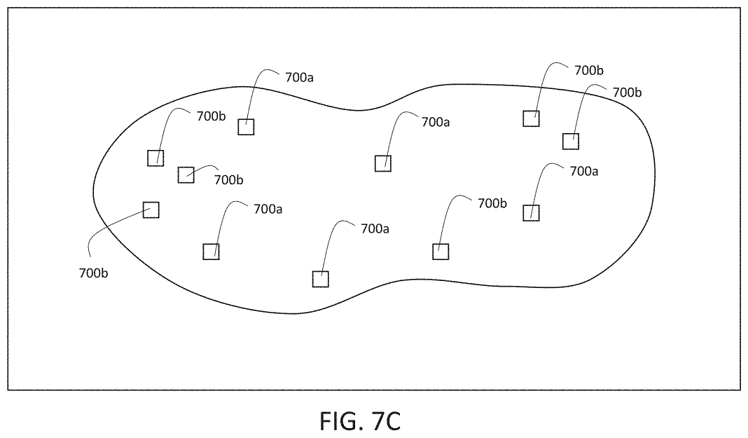

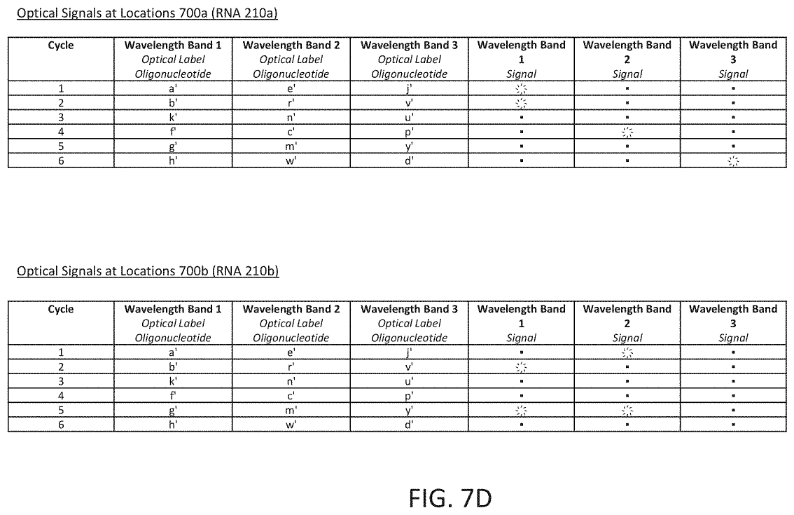

FIG. 7A is a schematic diagram showing an example illustrating the detection of a target RNA 210 in a sample via multiple cycles of hybridization and dehybridization of oligonucleotide-based, dye-containing optical labels, according to the workflow shown in FIG. 5. RNA 210 is bound to capture moiety 222, which is linked to a detection moiety 224 that includes reporter moieties 226a-226d. Different labeling agents are introduced into the sample in proximity to the reporter moieties 226a-226d. Each of the labeling agents includes an oligonucleotide sequence that corresponds to the oligonucleotide of a different one of the reporter moieties. In FIG. 7A, a first labeling agent 702a is deposited in the sample in proximity to reporter moiety 226a, and contains an oligonucleotide sequence that is associated with reporter moiety 226a. Second, third, and fourth labeling agents 702b, 702c, and 702d are also deposited in the sample in proximity to reporter moieties 226b, 226c, and 226d, respectively, and contain oligonucleotide sequences that are associated with reporter moieties 226b, 226c, and 226d, respectively.

Each of the labeling agents 702a-702d includes a distinct oligonucleotide sequence, and the respective sequences are labeled a, b, c, and d for reference. For an oligonucleotide sequence designated x, a partially or fully complementary sequence that hybridizes to sequence x is designated x′. Thus, for example, sequence a′ is complementary to and hybridizes to sequence a, sequence b′ is complementary to and hybridizes to sequence b, and so forth.

To detect RNA 210, multiple detection cycles are performed as described above in connection with FIG. 5. In each cycle, one or more optical labels with oligonucleotide sequences are introduced into the sample. Optical labels with sequences that are at least partially complementary to sequences of labeling agents 702a-d hybridize to the corresponding labeling agents. Measurement of the signals generated by the hybridized optical labels reveals the presence of the labeling agents at particular spatial locations in the sample. Co-location in the sample of signals attributable to each of the labeling agents associated with reporter moieties of the particular detection moiety of the probe 220 that binds to target RNA 210 reveals the presence of RNA 210 in the sample at that location.

In general, within each detection cycle, each different type of optical label includes a different oligonucleotide sequence, and generates a distinguishable optical signal. As such, within each cycle, following hybridization of one of more of the optical labels to complementary labeling agents, the optical signals corresponding to the different types of optical labels can be measured and distinguished, identifying the different types of optical labels that have hybridized to complementary labeling agents associated with different reporter moieties.

Optical signals generated by the different types of optical labels can be distinguishable in various ways. In some embodiments, for example, different types of optical labels include different dyes (e.g., fluorescent dyes) that emit light in different wavelength bands (i.e., bands that have different central wavelengths and/or different wavelengths of maximum emission intensity). If the wavelength bands are sufficiently separated spectrally, the optical signals can be distinguished from one another by spectral filtering and/or related techniques.

For optical signals that are not as well separated spectrally (e.g., when relatively higher numbers of different types of optical labels are used in a detection cycle), or that have non-Gaussian emission band shapes, a variety of computational techniques can be used to separate measured optical signals into component signals that individually correspond substantially to only one of the optical labels. For example, eigenvector decomposition methods such as spectral unmixing can be used to decompose multispectral images of a sample into a set of single-component images, each corresponding to spectral contributions from only one of the different types of optical labels.

To detect RNA 210 in the sample, individual labeling agents associated with each of the reporter moieties 226a-226d in FIG. 7A are detected in different cycles of the detection sequence. For purposes of illustration, within each detection cycle in FIG. 7A, three different types of optical labels are introduced. Each of the different types of optical labels includes a different oligonucleotide, and a fluorescent moiety that generates an optical signal in one of three different wavelength bands. Optical signals in each of the three different wavelength bands are distinguishable from one another using the methods discussed above.

It should be noted that the three different wavelength bands can be the same or different in different detection cycles. In some embodiments, for example, three different types of optical labels are introduced in each detection cycle, and each of the different types of optical labels generates an optical signal in only one of three different wavelength bands, with the three different wavelength bands remaining the same among all cycles. In other words, the same set of three different wavelength bands is re-used in each detection cycle.

In certain embodiments, some or all of the wavelength bands in which the different types of optical labels generate optical signals are different from one cycle to the next. That is, some of wavelength bands may be re-used from one cycle to the next, or none of the wavelength bands may be re-used. In general, there are no constraints on the manner in which different wavelength bands can be re-used or not re-used, except that within a particular detection cycle, each of the different types of optical labels generates an optical signal that is distinguishable from optical cycles generated by other types of optical labels within the cycle.

It should also be used that the introduction of three different types of optical labels in each detection cycle in FIG. 7A is merely an example for illustrative purposes. More generally, any number of different types of optical labels can be introduced in each cycle. The number of different types of optical labels that can be introduced can be the same among all cycles, or can vary among the cycles. Further, in some embodiments, each type of optical label is introduced only once among all detection cycles, as shown in FIG. 7A. More generally, however, some or all of the optical labels can be introduced in more than one (e.g., two or more, three or more, four or more, five or more, or even more) of the detection cycles. Introducing one or more of the optical labels in multiple detection cycles can allow verification of previously-detected optical signals that have been attributed to specific optical labels, for example.

The table in FIG. 7A shows an example of several cycles of optical label hybridization and measurement to detect RNA 210 in the sample. In each cycle, a first type of optical label generates an optical signal in Wavelength Band 1, a second type of optical label generates an optical signal in Wavelength Band 2, and a third type of optical label generates an optical signal in Wavelength Band 3. It should be noted that Wavelength Bands 1, 2, and 3 may be the same from one cycle to the next, or some or all of the Wavelength Bands may differ between any two cycles. The indicators “1”, “2”, and “3” designate only that the Wavelength Bands are different, and optical signals generated in each Wavelength Bands are distinguishable to allow identification of the optical labels that generate the signals, and therefore, the complementary labeling agents to which they are hybridized.

In cycle 1, optical labels with respective sequences a′, e′, and j′ are introduced into the sample. The first type of optical label with sequence a′ generates an optical signal in Wavelength Band 1, the second type of optical label with sequence e′ generates an optical signal in Wavelength Band 2, and the third type of optical label with sequence j′ generates an optical signal in Wavelength Band 3. Only the optical label with sequence a′ hybridizes to labeling agent 702, which corresponds to reporter moiety 226a. Thus, in cycle 1, a positive signal (i.e., a non-zero optical signal) is measured at the location of RNA 210 in the sample in Wavelength Band 1. No positive signals are measured in either Wavelength Band 2 or Wavelength Band 3.

In cycle 2, optical labels with respective sequences b′, r′, and v′ are introduced. The first type of optical label with sequence b′ generates an optical signal in Wavelength Band 1, the second type of optical label with sequence r′ generates an optical signal in Wavelength Band 2, and the third type of optical label with sequence v′ generates an optical signal in Wavelength Band 3. Only the optical label with sequence b′ hybridizes to labeling agent 702b, which corresponds to reporter moiety 226b. Therefore, in cycle 2, a positive signal is measured at the location of RNA 210 in the sample in Wavelength Band 1. No positive signals are measured in either Wavelength Band 2 or Wavelength Band 3.

The different types of optical labels introduced in cycles 3-6 are shown in FIG. 7A as well. Positive signals are measured at the location of RNA 210 in cycles 4 (in Wavelength Band 2 but not in Wavelength Bands 1 or 3) and 6 (in Wavelength Band 3 but not in Wavelength Bands 1 or 2). No positive signals are measured in any of the Wavelength Bands in cycles 3 or 5, as none of the optical labels in those cycles hybridizes to any of the labeling agents 702a-702d associated with the reporter moieties 226a-226d of detection moiety 224, which binds specifically to target RNA 210. The combination of positive signals measured in cycles 1 (in Wavelength Band 1), 2 (in Wavelength Band 1), 4 (in Wavelength Band 2), and 6 (in Wavelength Band 3), arising from optical labels with oligonucleotide sequences a′, b′, c′, and d′ in cycles 1, 2, 4, and 6, respectively, directly identifies that sequences a, b, c, and d corresponding to labeling agents 702a-702d are present in the sample, and these labeling agents are co-located.

The combination of labeling agent sequences a-b-c-d—the set of sequences of labeling agents 702a-702d—is uniquely associated with the combination of reporter moieties 226a-226d in detection moiety 224. In turn, detection moiety 224 is uniquely associated with target RNA 210, via the specific binding interaction between probe 220 and RNA 210. Accordingly, co-location of optical signals attributable to labeling agents 702a-702d in the sample allows the spatial distribution of RNA 210 in the sample to be determined and quantified.

It should also be noted that the foregoing methods do not distinguish among different orderings of reporter moieties within a detection moiety. In the example of FIG. 7A, by performing the 6 detection cycles shown, it can be determined that RNA 210 is present in sample by association of the measured signals with reporter moieties 226a-226d. The same set of reporter moieties 226a-226d would be identified, however, regardless of the particular ordering of the reporter moieties relative to capture moiety 222 in detection moiety 224. Any ordered, non-repeating combination of oligonucleotide sequences corresponding to reporter moieties 226a-226d yields the same identification of RNA 210.

Furthermore, it should be noted that the particular oligonucleotide sequences of labeling agents (i.e., a, b, c, and d) and their associations with the reporter moieties do not affect the identification of RNA 210, provided that each of sequences a, b, c, and d is uniquely associated with only one of reporter moieties 226a-d. There is no particular significance to the correspondence between the associations, provided that each of reporter moieties 226a-226d can be identified by measuring an optical signal that is, in some manner, attributable to only one of labeling agents 702a-d, and therefore, to only one of sequences a, b, c, and d.

In the example of FIG. 7A, in each set of optical labels that is introduced in each of the 6 detection cycles, at most one of the optical labels hybridizes to one of the labeling agents that has been deposited in the sample. However, as discussed above, more generally the sets of optical labels are selected such that in one or more of the detection cycles, multiple different optical labels can hybridize to individual labeling agents deposited in the sample, and generate optical signals that are detected and used to identify corresponding reporter moieties. By selecting the sets of optical labels in this manner, the overall efficiency of the assay can be increased by making increased use of the available detection channels during each detection cycle.

The example shown in FIG. 7A is representative, and illustrates the detection of a single RNA 210. In general, however, a sample includes many different RNAs, and the methods, compositions, and kits described herein are used to detect many different RNAs. For example, in some embodiments, 10 or more (e.g., 20 or more, 30 or more, 40 or more, 50 or more, 60 or more, 70 or more, 80 or more, 100 or more, 120 or more, 140 or more, 160 or more, 200 or more, 300 or more, 400 or more, 500 or more, 700 or more, 1000 or more, 1500 or more, 2000 or more, 3000 or more, 5000 or more, 7000 or more, 10000 or more, 15000 or more, 20000 or more, 30000 or more, 40000 or more, 50000 or more, or even more) different RNAs are detected in a sample using the methods, compositions, and/or kits described herein.

In the foregoing examples, probe 220 is implemented as a single molecular entity. More generally, however, probe 220 can be implemented as a single molecule, or as multiple molecules. For example, FIG. 4E shows an example of a probe 220 that consists of two probe portions. A first probe portion includes a first capture moiety 222p that selectively hybridizes to a first portion of target RNA 210, and a second probe portion includes a second capture moiety 222q that selectively hybridizes to a second portion of target RNA 210. Reporter moieties 226c and 226d are linked to first capture moiety 222p, while reporter moieties 226a and 226b are linked to second capture moiety 222q.

The methods, systems, kits, reagents, and other aspects of the present disclosure can each be implemented when probe 220 consists of a single molecular entity (see FIG. 2A), or when probe 220 consists of multiple molecular entities as in FIG. 4E. In general, probe 220 can include one or more (e.g., two or more, three or more, four or more, five or more, or even more) molecular entities, forming a combination of probes that functions as probe 220.

All of the features and aspects described herein in connection with probes, capture moieties, and reporter moieties, apply as well to multiple molecular entities that function together as probe 220.

As shown in FIG. 4E, when probe 220 consists of multiple molecular entities, the capture moieties (e.g., capture moieties 222p and 22q) bind to different portions of target RNA 210. In some embodiments, the capture moieties bind to adjacent and contiguous portions of the nucleotide sequence of target RNA 210, so that gap 225 spans no nucleotides of target RNA 210. In certain embodiments, the capture moieties bind to non-adjacent/non-contiguous portions of target RNA 210, so that gap 225 spans one or more (e.g., two or more, three or more, five or more, seven or more, 10 or more, 15 or more, 20 or more, 25 or more, 30 or more, 40 or more, 50 or more, or even more) nucleotides of target RNA 210.

It should also be noted that while in the example of FIG. 4E the reporter moieties 226a-226d are evenly distributed between the two molecular entities, more generally the reporter moieties can be evenly or unevenly distributed. In some embodiments, for example, one or more of the molecular entities that forms probe 220 can have a different number of reporter moieties than one or more of the other molecular entities that forms probe 220. In general terms, for a probe 220 that includes a total of P probes distributed among M molecular entities (each of which includes a capture moiety), each of the molecular entities can include a minimum of 1 reporter moiety, and a maximum of (P−M+1) reporter moieties.

When a sample contains multiple different RNAs, each different type of RNA selectively binds to a different type of probe or combination of probes that include a capture moiety specific to that type of RNA, and a detection moiety linked to the capture moiety that includes one or more reporter moieties, where the combination of reporter moieties in the detection moiety is specifically associated with that type of RNA. The multi-cycle detection methods described herein are used to detect the multiple different types of RNAs. During each detection cycle, the different optical labels introduced in that cycle can hybridize to complementary labeling agents deposited in the sample and associated with the reporter moieties of the different probes that are specific to the different types of RNAs. In general, in each cycle, the set of optical labels that are introduced can be selected so that multiple optical labels hybridize to one or more of the different types of reporter moieties, which ensures that individual detection cycles are efficiently used to elucidate the reporter moieties—and therefore the detection moieties—that are present in each location of the sample.

FIG. 7B shows two probes that specifically bind to two different RNAs in a sample. Specifically, the first probe includes a capture moiety 222a that specifically binds to a first type of RNA 210a, and is linked to a detection moiety 224a that includes reporter moieties 226a-226d. The second probe includes a capture moiety 222b that specifically binds to a second type of RNA 210b, and is linked to a detection moiety 224b that includes reporter moieties 226e-226h.

A first labeling agent 702a is deposited in the sample in proximity to reporter moiety 226a, and contains an oligonucleotide sequence that is associated with reporter moiety 226a. Second, third, fourth, fifth, sixth, seventh, and eighth labeling agents 702b, 702c, 702d, 702e, 702f, 702g, and 702h are also deposited in the sample in proximity to reporter moieties 226b, 226c, 226d, 226e, 226f, 226g, and 226h, respectively, and contain oligonucleotide sequences that are associated with reporter moieties 226b, 226c, 226d, 226e, 226f, 226g, and 226h, respectively. Each of the labeling agents 702a-702h includes a distinct oligonucleotide sequence, and the respective sequences are labeled a, b, c, d, e, f, g, and h for reference, in a manner analogous to the example of FIG. 7A described above.

FIG. 7C is a schematic image of the sample containing RNAs 210a and 210b. In the sample, RNA 210a is present at locations 700a, while RNA 210b is present at locations 700b. It should be noted that only one type of RNA is present at each physical location in the sample. In other words, two RNA molecules in the sample are not spatially coincident anywhere in the sample. Instead, each RNA molecule is present at a distinct location, although particular pairs of RNA molecules may be very closely spaced within the sample. The methods described herein can be used no matter how closely spaced two RNA molecules are in the sample.

FIG. 7D shows the measured optical signals from the sample at locations 700a and 700b over 6 detection cycles in tabular form. The upper table shows the measured optical signals at locations 700a (where RNA 210a is located in the sample). The lower table shows the measured optical signals at locations 700b (where RNA 210b is located in the sample). In each table, the sunburst character indicates that an optical signal was measured in a particular wavelength band, and the solid square character indicates that no optical signal was measured in a particular wavelength band.

As shown in column 1 of each table, 6 detection cycles were performed. Three optical labels were introduced in each detection cycle. The oligonucleotide sequences of each of the optical labels in each detection cycle, and the wavelength bands in which they generate optical signals, are shown in columns 2-4 of each table. Because the detection cycles were performed on the whole sample 10, columns 1-4 are identical in both tables.

The upper table indicates that positive optical signals were measured at locations (e.g., pixels) 700a in the sample in the following ordered pairs: (cycle 1, wavelength band 1), which corresponds to an optical label with oligonucleotide sequence a′; (cycle 2, wavelength band 1), which corresponds to an optical label with oligonucleotide sequence b′; (cycle 4, wavelength band 2), which corresponds to an optical label with oligonucleotide sequence c′; and (cycle 6, wavelength band 3), which corresponds to an optical label with oligonucleotide sequence d′. These positive optical signals are measured because reporter moiety 226a, which was exclusively present at locations 700a in the sample due to binding of capture moiety 222a to RNA 210a, is associated with labeling agents 702a-702d, which have oligonucleotide sequences a, b, c, and d, respectively.

The lower table indicates positive signals that were measured at locations (e.g., pixels) 700b in the sample. Reporter moiety 226b was exclusively present at locations 700b in the sample due to binding of capture moiety 222b to RNA 210b, and is associated with labeling agents 702e-702h, which have oligonucleotide sequences e, m, b, and g. The positive signals measured at locations 700b in the sample are represented by the following ordered pairs: (cycle 1, wavelength band 2), which corresponds to an optical label with oligonucleotide sequence e′; (cycle 2, wavelength band 1), which corresponds to an optical label with oligonucleotide sequence b′; (cycle 5, wavelength band 1), which corresponds to an optical label with oligonucleotide sequence g′; and (cycle 5, wavelength band 2), which corresponds to an optical label with oligonucleotide sequence m′.

Following the 6 detection cycles shown in FIG. 7D, each of the image pixels corresponding to sample locations 700a is associated with positive signals for labeling agents with oligonucleotide sequences a, b, c, and d, i.e., oligonucleotide sequences 702a-702d. Because only detection moiety 224a (via its combination of reporter moieties 226a-226d) corresponds to that combination of labeling agents, the presence of RNA 210a at locations 700a can be unambiguously identified.

Similarly, each of the image pixels corresponding to sample locations 700b is associated with positive signals for labeling agents with oligonucleotide sequences e, b, g, and m, i.e., oligonucleotides sequences 702e-702h. Because only detection moiety 224b (via its combination of reporter moieties 226e-226h) corresponds to that combination of labeling agents, the presence of RNA 210b at locations 700b can be unambiguously identified.

As is apparent from the results of cycle 2, when two different probes that bind to two different target RNAs contain the same reporter moiety, populations of the same labeling agent will be deposited in the sample at locations in proximity to the two different RNAs. This occurs in cycle 2, where a labeling agent with oligonucleotide sequence b (i.e., labeling agents 702b and 702g in FIG. 7B, which are the same labeling agent) is present in the sample at locations 700a and 700b. Optical signals are measured from optical labels that hybridize to each of the different labeling agents in a detection cycle in which the complementary optical label (e.g., an optical label with oligonucleotide sequence b′) is introduced.

However, the detection moieties are associated with different RNAs due to the specific nature of the interaction between the capture moieties and the RNAs. In the present example, detection moiety 224a is associated only with RNA 210a by virtue of the specific interaction between capture moiety 222a and RNA 210a, and detection moiety 224b is associated only with RNA 210b by virtue of the specific interaction between capture moiety 222b and RNA 210b. As a result, optical signals corresponding to detection moiety 224a will be measured only at locations 700a, and optical signals corresponding to detection moiety 224b will be measured only at locations 700b, even though the optical signals are generated by the same optical label with oligonucleotide sequence b′. In other words, because RNAs 210a and 210b are at spatially distinct locations in the sample, the measured optical signals corresponding to the optical label with sequence b′ have different meanings. Signals measured at locations 700a correspond to detection moiety 224a and RNA 210a, while signals measured at locations 700b correspond to detection moiety 224b and RNA 210b.

In general, it is not known a priori where any of the RNAs are located within the sample. Instead, the locations of particular types of RNAs are determined from the accumulated optical signal measurements at each pixel location. Specifically, each location in a sample (which corresponds to a particular pixel in the sample images that are obtained for detection of optical signals) is associated with a set of measured optical signals generated by optical labels during the multiple cycles of detection. At each location/pixel, the set of measured optical signals is associated with certain optical labels that were introduced during the detection cycles. Because the oligonucleotide sequences of the optical labels were known, the set of measured optical signals at each location/pixel is associated with populations of labeling agents at that location/pixel.

Each different type of detection moiety contains a unique combination of reporter moieties, and as explained previously, a unique combination of populations of labeling agents is therefore deposited in the sample at locations in proximity to the type of RNA to which the detection moiety specifically binds. Consequently, the populations of labeling agents at a particular location/pixel uniquely associates that location/pixel with a particular detection moiety, and therefore, a particular RNA at that location/pixel. In this manner, at each location/pixel within the sample, the presence of any one of the RNAs can be detected. Referring again to the example in FIGS. 7B-7D above, the co-location of labeling agents with oligonucleotide sequences a, b, c, and d at locations 700a in the sample identifies RNA 210a at locations 700a. Similarly, the co-location of labeling agents with oligonucleotide sequences e, m, b, and g at locations 700b in the sample identifies RNA 210b at locations 700b.

It should be noted that the foregoing procedure for identifying different RNAs is not sensitive to the ordering of reporter moieties in a detection moiety. That is, labeling agents are deposited in the sample in proximity to a corresponding target RNA in a manner that does not depend on the specific arrangement of reporter moieties in the detection moiety of the probe that specifically binds to the target RNA. As a consequence, for example, RNA 210a is identified at locations 700a in the sample no matter the relative ordering of reporter moieties 226a-226d in detection moiety 224a, and no matter the order in which labeling agents 702a-702d are deposited in the sample.

For locations/pixels at which no labeling agents are present (i.e., no optical signals are measured), no RNA is located at those locations/pixels. For locations/pixels at which an incomplete or erroneous set of populations of labeling agents is identified, the presence or absence of an RNA and the nature of the RNA at those locations/pixels is indeterminate if the determined set of populations of labeling agents cannot be corrected.

As discussed previously, in some embodiments, it can be advantageous to introduce one or more optical labels in two or more of the detection cycles in a multi-cycle detection procedure. For example, referring again to the example of FIG. 7A, after cycle 6, an additional detection cycle 7 can be performed in which one or more of the optical labels previously introduced is introduced again. By way of example only, consider a detection cycle in which optical labels with oligonucleotide sequences a′ (associated with wavelength band 1), s′ (associated with wavelength band 2), and q′ (associated with wavelength band 3) are introduced. The optical labels corresponding to sequences s′ and q′ will not hybridize to labeling agents present in the sample, and therefore generate no measured signal in any of the wavelength bands. The optical labels corresponding to sequence a′ will hybridize again to labeling agent 702a, and generate optical signals in wavelength band 1.

The signals are detected, as described above, by obtaining one or more images of the sample. Nominally, the spatial distribution of signals corresponding to the optical label with sequence a′ should be the same as the spatial distribution of signals corresponding to the optical label with sequence a′ in cycle 1. Accordingly, the measured optical signals generated by the optical labels with sequence a′ at each of the locations/pixels in cycle 7 can be used as a verification of the optical signals corresponding to optical labels with sequence a′ that were measured in cycle 1. Discrepancies may be corrected, or pixels at which discrepancies are observed can be marked and omitted from further analysis.

Thus, while introducing optical labels in more than one detection cycle can increase the time over which the assay is performed, doing so can also provide verification of previously measured optical signals, which can be an important consideration when the sample is of poor quality and/or certain RNAs are not easily or reproducibly bound to capture moieties. It should be noted that the introduction of optical labels in more than one detection cycle is advantageous when optical labels are dehybridized following measurement of optical signals. If the optical labels are instead deactivated or only partially removed, later re-introduction of the same optical labels may be more difficult.

In the foregoing discussion, detection moiety 224 of each type of probe 220 includes a combination of reporter moieties 226 that is unique to that type of probe 220. In practice, other constraints can be applied to the combinations of reporter moieties 226 in each type of probe 220 to further ensure that particular types of analytes to which the probes are selectively bound are detected, and to correct for errors that can occur during binding (e.g., hybridization) of the catalytic agents and/or labeling agents, and imaging of the bound or associated optical labels. Errors that can occur during binding can include, but are not limited to: incomplete hybridization of catalytic agents; incomplete hybridization of labeling agents; to inadvertent dehybridization of labeling agents and/or catalytic agents during, or prior to, measurement of optical signals; cross-hybridization of catalytic agents and/or labeling agents; and incomplete removal of catalytic agents and/or labeling agents at the end of a cycle. Errors that can occur during imaging include, but are not limited to: obscuration of optical signals generated by optical labels (e.g., obscuration by sample features or conformational changes of the reporter moieties and/or optical labels); inadvertent quenching of optical signals generated by optical labels by other sample features; and loss of resolution due to non-specific localization of optical labels.

In general, constraints on the choice of reporter moieties among different types of probes 220 can be applied to correct for a number of different types of errors that can arise when labeling analytes and measuring corresponding optical signals from the labeled analytes. These include, for example, reducing or eliminating erroneous hybridization of catalytic and/or labeling agents. Hybridization of such an agent to an oligonucleotide to which it is not intended to hybridize, but to which a certain extent of inadvertent complementarity nonetheless exists, is generally not corrected in this manner. However, erroneous spatial localization of a catalytic and/or labeling agent in the vicinity of an analyte that occurs once in anomalous fashion can be determined and corrected, as can the absence of such localization in the vicinity of an analyte that occurs once, when such localization would have been expected.

Errors that can be corrected can also include incorrect assignment of individual regions (which may correspond to voxels in a sample) that arise when different optical labels generate optical signals that are spectrally similar. When measured signals from different optical labels are sufficiently similar, it can be challenging to assign spatial regions associated with the signals to the correct labeled analyte. Such errors can be reduced or minimized, for example, by distributing optical labels among multiple fluorescence channels (i.e., selecting the optical labels such that they have fluorescence emission spectra that are sufficiently different), such that optical signals measured from the different optical labels can be readily distinguished.

Additional errors that can be corrected through selection of appropriate combinations of optical labels, reporter moieties, and the use of multi-cycle detection methodologies include the absence of detectable optical signals when such signals are expected to be present. Expected optical signals that are not measured can result in the absence of a measured optical signal that would otherwise be used for identification of a labeled analyte. By appropriate selection of optical labels, reporter moieties, and the use of multi-cycle detection methodologies, the absence of expected measured optical signals during detection can be corrected, ensuring that labeled analytes in the sample can still be identified.

To provide for correction of errors during identification of multiple analytes in a sample labeled with probes that include different combinations of reporter moieties, the individual reporter moieties are selected according to certain constraints, and the optical labels that are used in the multi-cycle detection methodology described above are also selected appropriately to ensure that individual labeled analytes can still be distinguished from one another, even when certain types of detection errors occur during the multi-cycle detection methodology.

To further describe the selection of reporter moieties and optical labels in connection with multi-cycle detection methodologies, the following terminology is used. In general, each probe 220 includes R different reporter moieties. For R=4, as shown in FIG. 2C, the label regions are 226a-226d. More generally, however, detection moiety 224 can include any number R of reporter moieties, such that R can be 2 or more (e.g., 3 or more, 4 or more, 5 or more, 6 or more, 7 or more, 8 or more, 9 or more, 10 or more, or even more).

Typically, each different type of analyte in the sample that is analyzed is labeled with a different type of probe 220, i.e., a probe 220 with a different combination of reporter moieties. In some embodiments, each of the different types of probes includes the same number R of reporter moieties. In certain embodiments, different types of analytes are labeled with different types of probes 220, and among the different types of probes, one or more have different numbers of reporter moieties than the other types of probes. For example, among the different types of probes, there can be one or more (e.g., 2 or more, 3 or more, 4 or more, 5 or more, 6 or more, 7 or more, 8 or more, 9 or more, 10 or more, or even more) groups of different types of probes, each group having a different number of reporter moieties. Each such group can include one or more (e.g., 2 or more, 3 or more, 4 or more, 5 or more, 6 or more, 7 or more, 8 or more, 9 or more, 10 or more, or even more) reporter moieties.

Each reporter moiety can consist of multiple nucleotide bases that form a nucleotide sequence. In some embodiments, each of the R reporter moieties within a detection moiety has the same number of nucleotide bases. In certain embodiments, one or more of the R reporter moieties consists of a first number of nucleotide bases, and one or more of the R reporter moieties consists of a second number of nucleotide bases that is different from the first number of nucleotide bases. In general, any of the R reporter moieties within a detection moiety can include any number of nucleotide bases.

Among multiple probes used to label different analytes in a sample, in certain embodiments the R reporter moieties in a first probe used to label a first analyte can each have the same number of nucleotide bases as the R reporter moieties in a second probe used to label a second analyte that is different from the first analyte. More generally, however, the number of nucleotides in one or more of the reporter moieties of a first probe can differ from the number of nucleotides in one or more corresponding label regions of a second probe. Each reporter moiety in any of the probes described herein can independently include 2 or more (e.g., 3 or more, 4 or more, 5 or more, 10 or more, 15 or more, 20 or more, 30 or more, 40 or more, 50 or more, 60 or more, 70 or more, 80 or more, 90 or more, 100 or more, 120 or more, 140 or more, 160 or more, 180 or more, 200 or more, or even more) nucleotide bases. The number of nucleotide bases in any reporter moiety can be selected, for example, to control the kinetics, binding strength, and stringency of hybridization.

The R reporter moieties in each probe 220 form a reporter code C. In general, each analyte in the sample is labeled with a different probe 220, i.e., a probe 220 with a detection moiety 224 that contains a unique combination of R reporter moieties forming a reporter code C. Thus, to detect T different analytes in a sample, a pool of probes with T unique reporter codes C is used. In particular, the sample is exposed to a composition that includes T unique probes, each of which specifically binds a different one of the analytes. All probes with the same type of reporter code C contain the same type of capture agent, so that multiple analytes of the same type in the sample are labeled with the same type of reporter code C. In this manner, each different type of analyte in the sample is labeled with a unique detection moiety having a unique reporter code C. Detecting each of the reporter codes C therefore allows each of the different types of analytes to be identified in the sample, and determining quantities of each of the different reporter codes C allows the different types of analytes to be quantified in the sample.

In general, the number T of unique reporter codes, which may be the same as the number of unique types of probes 220 in a composition to which the sample is exposed, and may also be the same as the number of unique analytes that are detected, can be selected as desired. For example, T can be 2 or more (e.g., 3 or more, 4 or more, 5 or more, 10 or more, 15 or more, 20 or more, 25 or more, 30 or more, 35 or more, 40 or more, 45 or more, 50 or more, 60 or more, 70 or more, 80 or more, 90 or more, 100 or more, 200 or more, 300 or more, 400 or more, 500 or more, 600 or more, 800 or more, 1000 or more, 1500 or more, 2000 or more, 2500 or more, 3000 or more, 3500 or more, 4000 or more, 5000 or more, 6000 or more, 7000 or more, 8000 or more, 9000 or more, 10000 or more, 15000 or more, 20000 or more, 30000 or more, 40000 or more, 50000 or more, or even more).

To understand the nature of errors during hybridization and detection of corresponding signals to identify analytes, consider a pool of N different oligonucleotide sequences. For purposes of illustration only, N can be assumed to be 26, with each of the oligonucleotide sequences being designated with a single-letter identifier from “a” to “z”. It should be noted that this value of N and the single-letter oligonucleotide sequences identifiers are used only by way of explanation, and in general, N can have any value, and the oligonucleotide sequences can be designated in any manner.

Consider further a particular reporter code C* having four regions with oligonucleotide sequences a-b-c-d. Reporter code C* is part of detection moiety 224, which is linked to capture moiety 222. Capture moiety 222, in turn, specifically binds to a particular analyte in the sample at spatial location L in the sample.

During multiple detection cycles, to detect the presence of reporter code C*, optical labels that are associated with oligonucleotide sequences a, b, c, and d are introduced into the sample at location L, and signals arising from these labels are detected at location L. Because reporter code C* is conjugated to a capture moiety 222 that specifically binds to a particular analyte Y, the presence of that analyte Y at location L can be identified.

In some embodiments, the T unique reporter codes C are selected such that each of the codes is distinguishable when a single drop error occurs during hybridization and detection of optical labels. In the foregoing example, optical signals corresponding to oligonucleotide sequences a, b, c, and d are expected to be measured at location L. A single drop error occurs when one of these optical labels is not measured at location L. Any one of the expected optical signals can be the missing signal. For example, measured optical signals at location L can correspond to sequences a, b, and d, and therefore the reporter code C* based on the measured optical signals at location L is a-b-d.

Under these circumstances, reporter code C* determined from multiple cycles of hybridization of optical labels and subsequent detection of emitted light from the hybridized labels is in error, as it only contains three label regions a, b, d, rather than four label regions. Without any restriction on the selection of the T unique reporter codes C that are conjugated to capture moieties within a pool of probes, it may not be possible to determine which analyte is located at location L in the sample. This ambiguity arises because it may not be possible to determine which additional oligonucleotide sequence (i.e., from among a-z) was present within reporter code C*, and was not detected.

To ensure that detected reporter codes C with a single drop error can still be distinguished from other reporter codes C in a composition of probes, the reporter codes can be selected according to particular constraints. In general, the composition of probes includes different types of probes, where each different type of probe includes a capture moiety 222 linked to a different type of detection moiety 224 that contains a different type of reporter code C. To ensure that reporter codes with single drop errors can be distinguished from other reporter probes, each type of reporter code C is selected such that it differs from each of the other types of reporter codes C in the composition by at least two reporter moieties, irrespective of the relative positions of the reporter moieties in the reporter codes.

Thus, for example, if a composition contains one type of reporter code a-b-c-d present in one type of detection moiety 224 that is linked to a capture moiety 222 that selectively binds a first type of analyte, then the composition does not contain any of the following examples of reporter codes in any different type of detection moiety 224 linked to a capture moiety 222 that selectively binds a second type of analyte different from the first type of analyte: a-b-c-e (because this code differs from a-b-c-d by only one reporter moiety), d-c-b-f (because this code differs from a-b-c-d by only one reporter moiety), b-g-d-a (because this code differs from a-b-c-d by only one reporter moiety), and c-a-h-b (because this code differs from a-b-c-d by only one reporter moiety). Among the foregoing examples, the order of the reporter moieties does not matter for purposes of determining the differences between reporter codes. Thus, d-c-b-f differs from a-b-c-d by only one reporter moiety (e.g., f has replaced a), even though the relative ordering of reporter moieties b, c, and d differs between the codes.

Because the relative ordering of the reporter moieties does not matter, the composition also does not contain any of the following examples of reporter codes in any different type of detection moiety: a-d-b-c, c-d-a-b, and b-a-d-c. Each of these reporter codes does not differ from a-b-c-d at all, because the relative ordering of the reporter moieties does not matter. In some embodiments, codes such as this—which differ only from a-b-c-d in the relative ordering of the reporter moieties—are not present at all in the composition. In certain embodiments, codes such as this may be present in the composition, but only as part of a detection that is linked to a capture moiety that selectively binds the first type of analyte.

It should be noted that the foregoing examples of reporter codes are not exhaustive, and the constraint discussed above excludes other reporter codes C from the detection moieties 224 of the composition as well.

With the above constraint applied to the selection of reporter codes C present in different types of detection moieties of different types of probes in the composition, detected reporter codes that result from a single-drop error can still be distinguished. For example, if a reporter code a-b-c-d is detected as a-b-d due to a single-drop error, then the detected code a-b-d can still be distinguished from other reporter codes of other types of probes, because the other reporter codes of other types of probes in the composition will not have the combination of a, b, and d label regions, since each reporter code differs from all other reporter codes linked to different types of capture moieties by at least two reporter moieties. In other words, a detected reporter code a-b-d that contains a single drop error can still be distinguished from reporter codes such as a-b-e-f, d-b-r-s, b-a-f-g, and g-h-c-d.

In some embodiments, the T unique reporter codes C are selected such that each of the codes is distinguishable when a single add error occurs during hybridization and detection of optical signals. Continuing the foregoing discussion, optical signals corresponding to optical labels that are associated with reporter moiety oligonucleotide sequences a, b, c, and d are expected to be measured at location L. A single add error occurs when an optical signal corresponding to an additional optical label is measured at location L. The additional optical signal can correspond to any one of the optical labels. For example, measured optical signals at location L can correspond to optical labels that are associated with reporter moiety oligonucleotide sequences a, b, c, d, and e, and therefore the reporter code C* based on the measured optical signals at location L is a-b-c-d-e.

To ensure that detected reporter codes C with a single add error can still be distinguished from other reporter codes C in a composition of probes, each type of reporter code C is selected such that it differs from each of the other types of reporter codes C in the composition by at least two label regions, irrespective of the relative positions of the reporter moieties in the reporter codes.

Referring to the example above, suppose in addition to reporter code a-b-c-d as part of a first probe, which is erroneously detected as a-b-c-d-e, the composition also contains reporter code a-b-e-f as part of a second probe, which differs from reporter code a-b-c-d by two reporter moieties (i.e., the third and fourth reporter moieties). If reporter code a-b-e-f is correctly detected, then the second probe can be distinguished from the first probe based on reporter moiety f In similar fashion, when reporter codes are present in probes of a composition according to the above constraint, each different type of probe—and the different analytes to which they selectively bind—can be distinguished, even when reporter codes are detected with single add errors.

In some embodiments, the T unique reporter codes C are selected such that the occurrence of a single replacement error detection of optical signals can be determined. Returning again to the discussion above, optical signals corresponding to optical labels that are associated with reporter moiety oligonucleotide sequences a, b, c, and d are expected to be measured at location L. A single replacement error occurs when an optical signal corresponding to an unexpected optical label is measured at location L, in place of an expected optical label signal. The replacement optical signal can be detected in place of any one of the optical labels. For example, measured optical signals at location L can correspond to optical labels associated with reporter moiety oligonucleotide sequences a, b, c, and e, and therefore the reporter code C* based on the measured optical signals at location L includes sequences a, b, c, and e, instead of sequences a, b, c, and d.

However, while a reporter code C corresponding to a combination of sequences a, b, c, and e can be recognized as invalid, the reporter code cannot necessarily be corrected, because it is not known which of the sequences a, b, c, and e was detected erroneously. That is, any one of sequences a, b, c, or e may have been the sequence that was detected in error. Thus, while the determined reporter code C that includes sequences a, b, c, and e can be identified as invalid, it may not be possible to determine the correct reporter code without additional information. Accordingly, detection signals corresponding to such reporter codes can simply be eliminated from further consideration on the grounds that they are erroneous.

To ensure that detected reporter codes C with a single replacement error can be identified as erroneous, each type of reporter code C can be selected such that it differs from each of the other types of reporter codes C in the composition by at least two reporter moieties, irrespective of the relative positions of the reporter moieties in the reporter codes.

In addition to single add, single drop, and single replacement errors, other types of detection errors specific to reporter codes can also occur. One such example is a double drop error, in which a detection moiety 224 contains Q reporter moieties, but only (Q−2) reporter moieties are detected. For example, detection moiety 224 can include 4 label regions a-b-c-d forming a reporter code C, but during detection, only label regions a and b are detected. Thus, the detected reporter code is a-b.

Another example is a double add error, in which a detection moiety 224 contains Q label regions, but (Q+2) label regions are detected. As an example, detection moiety 224 includes reporter moieties a-b-c-d forming reporter code C, but during detection, the reporter code is determined to be a-b-c-d-e-f.

In some embodiments, double add and/or double drop errors can be identified, but cannot be corrected without additional information. Consequently, as discussed above in connection with single replacement errors, corresponding detection signals can simply be eliminated from further consideration, on the grounds that they are erroneous.

In certain embodiments, double add and/or double drop errors can be corrected by applying constraints to the selection of reporter codes C as described above. For example, to be able to distinguish reporter codes of two detection moieties 224 that are linked to two different types of capture moieties, the reporter codes C in a composition can be selected according to the constraint that each type of reporter code C is selected such that it differs from each of the other types of reporter codes C in the composition by at least three label regions, irrespective of the relative positions of the reporter moieties in the reporter codes. When detection moieties in a composition differ by at least three reporter moieties, detection moieties that are subject to erroneous detection with a double add or double drop error can still be distinguished from other detection moieties in the composition.

In some embodiments, other constraints can be applied to the selection of reporter codes C to ensure that different types of detection moieties can be distinguished from one another. For example, reporter codes C can be selected such that each code does not contain any duplicate reporter moiety oligonucleotide sequence. In other words, each detection moiety 224 contains Q reporter moieties, and each of the Q reporter moieties is different in the detection moiety.

In some embodiments, additional calibration and error checking steps can be performed during multi-cycle hybridization and detection of optical labels. For example, to identify a reporter moiety that is part of a probe, an optical label that is associated with that reporter moiety is typically measured once during a multi-cycle hybridization and detection procedure. A single measurement of an optical signal from the optical label associated with the reporter moiety is generally sufficient to ensure that the reporter moiety is detected.

However, in some embodiments, one or more of the optical labels are measured more than once—either by obtaining more than one image after introducing the optical label once, or by introducing (and measuring signal from) the optical label in more than one cycle of the multi-cycle detection procedure. Such procedures can be performed as a verification of measured detection signals arising from the optical labels. For example, an initial image of a particular optical label in a sample yields a spatial distribution of optical signals corresponding to the optical label. By repeating measurement of the same optical signal in a later cycle, the measured spatial distribution of the optical signals can be verified and, if necessary, corrected based on the second measured spatial distribution of optical signals. Verification can be used both to ensure that spurious optical signals were not measured initially, and that optical signals that should have been measured initially were not undetected. By verifying the measured optical signals in such a manner, measurements of the spatial distributions of particular analytes can be confirmed. Furthermore, quantitative measurements of particular analytes, which are obtained by integrating spatial distributions of the analytes, can also be verified.

In certain embodiments, probes that include detection moieties can also include binding regions for optical labels that are not part of detection moieties, but instead are used to perform standardization or calibration functions. For example, the probes discussed herein can include includes one or more verification regions, and each of the verification regions generally includes an oligonucleotide sequence that can have some or all of the same properties as the reporter moieties discussed previously.

The sequences of each of the verification regions are generally selected such that they are not associated with any of the optical labels. As such, during detection of signals from optical labels, no measured signals are associated with the verification regions. However, the procedures described herein can be modified to include one or more standardization steps in which a standardization optical label, which generates an optical signal that is different from the optical signals generated by the optical labels that are associated with reporter moieties, is introduced into the sample and hybridizes to one of the verification regions of the probe. The standardization optical label generates a standardization optical signal which is detected in the same manner discussed previously.

In general, probes can include one or more (e.g., two or more, three or more, four or more, five or more, or even more) verification regions. Each of the verification regions in a probe can be different (e.g., can have a different oligonucleotide sequence), or alternatively, one or more of the verification regions can be the same as another of the verification regions in the probe.

In some embodiments, measured optical signals corresponding to particular groups or classes of analytes can be reliably distinguished from other optical signals in a multi-analyte assay by measuring signals corresponding to a standardization optical label that hybridizes to a standardization region of the probes. Further, and in particular by measuring optical signals arising from a standardization optical label that hybridizes to a standardization region that is present in all probes, optical signals measured in different portions of a sample can be corrected relative to one another. That is, the optical signal arising from the standardization optical label functions as a common reference optical signal across the sample, in which variations in the signal can be presumed to arise from sample inhomogeneity, variance in detection sensitivity, and other factors that may yield variations in imaging across a spatial field of view. Measured optical signals arising from optical labels can be corrected for such variations, using the optical signal from the standardization optical label as a reference signal. A wide variety of such corrections are possible, including but not limited to dividing optical signals by the corresponding standardization optical label signal (i.e., a spatially-resolved correction), normalizing or scaling optical signals to yield signals that deviate reproducibly from the reference standardization optical signal, and using the standardization optical signal to establish thresholds for positive and negative signal detection among the measured optical signals associated with reporter moieties.

### Reagents and Conditions

In general, the various steps described herein can be implemented under a wide variety of conditions and with different reagents. Accordingly, the reagents and conditions described in this section should be understood to represent only examples of suitable reagents and conditions.

Typically, probes can be stored following preparation in a buffer solution that can include one or more of PBS, PBS-T, TBS, TBS-T, water, saline solution, and Kreb's buffer. The buffer solution can optionally include one or more blocking materials, such as (but not limited to) oligonucleotides.

Catalytic agents can also be stored following preparation in a buffer solution. The buffer can include one or more of PBS, PBS-T, TBS, TBS-T, water, saline solution, and Kreb's buffer. The buffer solution can be the same as, or different from, the buffer solution used to store the probes.

To promote hybridization between the probes and catalytic agents, the probes and catalytic agents can be immersed in a hybridization buffer. Suitable hybridization buffers can include DNA components, protein components, detergents, and/or chaotropic reagents at concentrations of between 5% and 20%.

To promote de-hybridization between the probes and catalytic agents, the probes and catalytic agents can be immersed in a de-hybridization buffer. Suitable de-hybridization buffers can include chaotropic reagents such as DMSO and/or formamide, at concentrations of between 60% and 90%.

To promote binding of the probe to a target RNA in a sample, the probe can be layered onto the sample in solution, e.g., by pipetting, and incubated with the sample. Following incubation, unbound probes can be washed from the sample using, for example, a buffer solution that includes one or more of PBS, PBS-T, TBS, TBS-T, water, saline solution, and Kreb's buffer.

The incubation time for any of the hybridization, reaction, binding, and de-hybridization steps described herein can be 10 minutes or more (e.g., 20 minutes or more, 30 minutes or more, 40 minutes or more, 60 minutes or more, 1 hour or more, 2 hours or more, 3 hours or more, 4 hours or more, 5 hours or more, 6 hours or more, 8 hours or more, 10 hours or more, 16 hours or more, 20 hours or more, 24 hours or more, 48 hours or more, 7 days or more, 30 days or more).

### Error Correction

In the methods discussed above, no restrictions have been applied to the selection of reporter moieties in the detection moieties of probes for different RNAs, other than to note that each different type of probe (which specifically binds to a different type of RNA) contains a detection moiety with a unique combination of different reporter moieties, without regard to the ordering of the reporter moieties in the detection moiety (i.e., the uniqueness depends on the specific reporter moieties present in the detection moiety, not on their ordering within the detection moiety).

In some embodiments, various criteria and/or constraints can be applied to the distribution of the reporter moieties among the detection moieties of different types of probes, and error correction methods that are based on these criteria and/or constrains can be used to detect errors in measured optical signals in the sample that arise (or are presumed to arise) from optical labels that hybridize to the labeling agents that are associated with reporter moieties of the detection moieties. Correction methods can be applied to identify the absence of expected optical signals that are not measured in the sample. Certain types of errors can also be corrected to allow particular reporter moieties (and the detection moieties that contain them) to be identified even when measured optical signals are in error.

Examples of such criteria and constraints for the selection of reporter moieties, and methods, compositions, and kits for performing error correction, are described in U.S. patent application Ser. No. 17/714,628, the entire contents of which are incorporated herein by reference.

### Compositions and Kits

The probes, labeling agents, optical labels, species, and all moieties and other compounds and substances described herein can be included in a variety of kits featuring compositions that include the probes, labeling agents, optical labels, species, and moieties. In general, a kit is a package of one or more reagents, each of which is in the form of a composition. Compositions featuring any of the different probes, labeling agents, optical labels, species, and moieties described herein can be prepared and used for target analyte analysis as described herein. These compositions can be included in product kits, along with other features such as instructions for preparing compositions, and using the compositions for sample analysis. Product kits can be sealed or otherwise contained in a variety of different containers.

In some embodiments, a composition—to which a sample can optionally be exposed—can include a plurality of probes. Each of the probes can include a capture moiety 222 and a detection moiety 224. The detection moiety 224 can include one or more reporter moieties 226. Reporter moieties 226 can have any of the properties described herein. The probes can also optionally include any of the other features described herein.

In certain embodiments, the compositions can include any one or more of the species (i.e., probes, oligonucleotides, optical labels, and other species) described herein that are introduced into the sample. For example, the compositions can include any of the types of probes and other species described in connection with the workflows of FIGS. 1, 5 and 6.

Reporter moieties in a composition can be selected such that, when the compositions are used to label samples, different types of RNA in the sample are labeled with different types of probes. Each type of probe can selectively bind to a different type of RNA in the sample. Typically, probes of the same type each include the same combination of reporter moieties within the detection moieties of the probes, or optionally, reporter moieties that are indistinguishable variants of one another (i.e., reporter moieties that may vary slightly, but hybridize to the same catalytic agents, for example) so that the deposited populations of labeling agents that correspond to the detection moieties of the probes are the same. Probes of each type have detection moieties that differ from the detection moieties of probes of other types, without regard to the relative ordering of the reporter moieties in each type of probe.

Compositions can generally include probes, catalytic agents, labeling agents, species, and other components that are used to label a sample as described herein such that any number of different types of RNAs in the sample can be distinguishably labeled. In some embodiments, for example, compositions can be used to label 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, or even more) different types of RNA analytes in the sample, and can contain distinguishable probes, catalytic agents, labeling agents, oligonucleotides, and other components of the same number.

Each population or group of probes, catalytic agents, labeling agents, oligonucleotides, or another component within a composition can generally include any number of probes, catalytic agents, labeling agents, oligonucleotides, catalytic agents, labeling agents, and/or other component of a particular type. For example, the number of probes, catalytic agents, labeling agents, oligonucleotides, or another component of a particular type can be 1 or more (e.g., 2 or more, 3 or more, 5 or more, 10 or more, 50 or more, 100 or more, 200 or more, 300 or more, 500 or more, 1000 or more, 2000 or more, 3000 or more, 5000 or more, 7000 or more, 10000 or more, 30000 or more, 50000 or more, 100000 or more, 500000 or more, or even more).

Reporter moieties can be selected to yield different types of probes in the composition. Compositions can generally include 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, 20000 or more, 30000 or more, 50000 or more, or even more) different types of reporter moieties.

In general, compositions can include populations or groups of different types of detection moieties, each of which includes one or more reporter moieties of the same (or indistinguishable) type. In some embodiments, for example, compositions can include populations or groups of 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, or even more) different types of detection moieties.

Each population or group of a different type of detection moiety within a composition can generally include any number of detection moieties. For example, the number of detection moieties of a particular type can be 1 or more (e.g., 2 or more, 3 or more, 5 or more, 10 or more, 50 or more, 100 or more, 200 or more, 300 or more, 500 or more, 1000 or more, 2000 or more, 3000 or more, 5000 or more, 7000 or more, 10000 or more, 30000 or more, 50000 or more, or even more).

In some embodiments, compositions can include one or more additional components. For example, in some embodiments, compositions can include one or more buffer solutions. Examples of suitable buffer solutions include, but are not limited to, saline-sodium citrate (SSC), phosphate-buffered saline (PBS), and tris-ethylenediaminetetraacetic acid (tris- EDTA).

In certain embodiments, compositions include one or more optical labels. The optical labels can include any of the optical labels described herein. Typically, as described in connection with FIGS. 6 and 7A-7D, a sample is exposed to optical labels in groups or pools, where each such group includes 2 or more (e.g., 3 or more, 4 or more, 5 or more, 6 or more, 8 or more, or even more) different optical labels. Within a composition, the optical labels can optionally be divided into sub-compositions, each of which contains a group or pool of optical labels. In certain embodiments, among sub-compositions, each optical label can be present in only one sub-composition. Alternatively, in some embodiments, one or more optical labels can be present in more than one sub-composition.

The sample is exposed to a composition that includes optical labels to hybridize some or all of the optical labels to complementary labeling agents, as described above. In certain embodiments, the sample is exposed to the entire composition of optical labels at the same time. Alternatively, in some embodiments, the sample is exposed to sub-compositions of the composition sequentially as discussed previously.

In some embodiments, optical labels are present in the same composition as the probes, catalytic agents, labeling agents, oligonucleotides, and/or other components discussed above. In certain embodiments, optical labels are present in a different composition to which the sample is exposed after exposure to one or more compositions that include(s) the probes, catalytic agents, labeling agents, oligonucleotides, and/or reporter moieties.

Compositions that include optical labels can also optionally include additional components. For example, in some embodiments, compositions can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

One or more of the compositions described above can be included as part of a kit for analyzing a sample. The kit can include a housing or packaging that encloses the contents of the kit. Compositions can be contained within containers in the kit; such containers can be formed from a wide variety of materials, including (but not limited to) plastics and glass.

Kits can optionally include a variety of other components as well. For example, in some embodiments, kits can include one or more dehybridization reagents. Examples of such reagents include, but are not limited to, sodium hydroxide, dimethyl sulfoxide (DMSO), formamide, SDS, methanol, and ethanol.

In certain embodiments, kits can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

Kits can also optionally include instructions printed or otherwise recorded on any of a variety of different media (e.g., paper, computer readable storage media) that describe the use of certain components of the kits in assays targeting RNAs of various types in samples.

### Applications

The methods, compositions, and kits described herein can be used to detect many different types of RNAs in a wide variety of different samples. The methods, compositions, and kits do not depend on the manner in which RNAs in the sample bind to probes, nor do they depend on the nature of the RNAs. Further, other than the aspects of the probes described herein that are related to steps in workflow, probes can have a wide variety of different structural compositions and functionalities. Provided they do not interfere with the deposition of labeling agents and/or hybridization of optical labels, additional structural features of the probes can be present without interfering with the methods described herein.

Samples to which the methods, compositions, and kits can be applied include tissue samples (e.g., tissue sections), including fresh, fresh-frozen, and formalin-fixed, paraffin-embedded (FFPE) samples. Other samples include, but are not limited to, cell and tissue lysates, body fluids (e.g., blood, urine, saliva, lymph fluid, renal fluid, and other such fluids), thick tissue (e.g., solid tumor tissue, biopsy samples), individual cells, suspensions of cells and other biological materials, and aspirated samples.

Additional applications and aspects of the methods and systems disclosed herein are described, for example, in U.S. patent application Ser. No. 17/714,628, the entire contents of which are incorporated herein by reference.

### Analysis Systems and Components

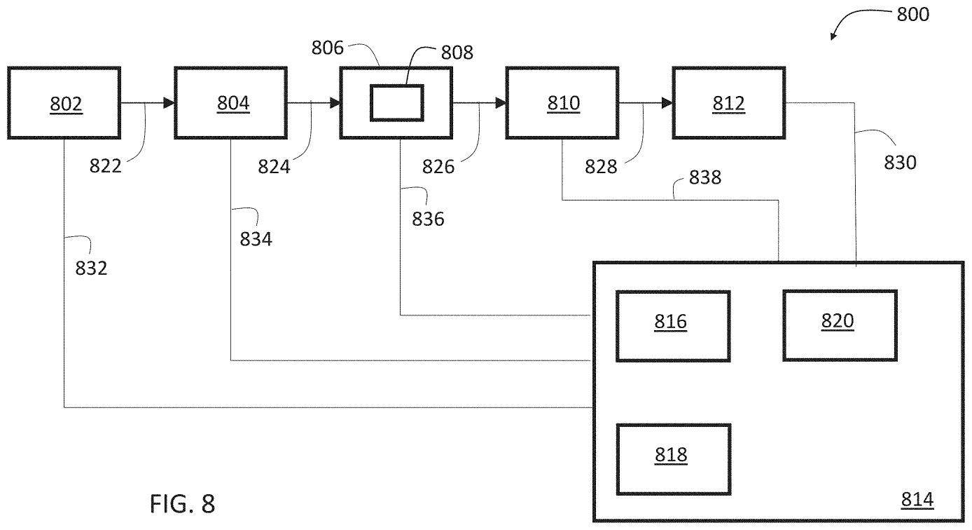

FIG. 8 is a schematic diagram showing a system 800 for acquiring multiple spectrally resolved images of a sample. System 800 can measure light emitted from, transmitted from, and/or reflected by a sample that includes one or more of the labeling agents and/or optical labels described herein. The measured light generally includes contributions from each of the labeling agents and/or optical labels present in the sample, and system 800 can analyze the multispectral image information encoded in the measured light, decomposing the image information to isolate contributions to the measured light from each of the labeling agents and/or optical labels in the sample. The decomposition yields, for each labeling agent or optical label in the sample, a set of amplitude or intensity measurements as a function of position within the sample. The amplitude or intensity measurements can be used to quantify the amount of each labeling agent or optical label, and therefore the amount of each target analyte, and each position in the sample.

A light source 802 provides light 822 to light conditioning optics 804. Light 822 can be incoherent light, such as light generated from a filament source for example, or light 822 can be coherent light, such as light generated by a laser. Light 822 can be either continuous-wave (CW) or time-gated (i.e., pulsed) light. Further, light 822 can be provided in a selected portion of the electromagnetic spectrum. For example, light 822 can have a central wavelength and/or a distribution of wavelengths that falls within the ultraviolet, visible, infrared, or other regions of the spectrum.

Light conditioning optics 804 can be configured to transform light 822 in a number of ways. For example, light conditioning optics 804 can spectrally filter light 822 to provide output light in a selected wavelength region of the spectrum. Alternatively, or in addition, light conditioning optics can adjust the spatial distribution of light 822 and the temporal properties of light 822. Incident light 824 is generated from light 822 by the action of the elements of light conditioning optics 804.

Incident light 824 is directed to be incident on sample 608 mounted on illumination stage 806. Stage 806 can provide means to secure sample 808, such as mounting clips or other fastening devices. Alternatively, stage 806 can include a movable track or belt on which a plurality of samples 808 are affixed. A driver mechanism can be configured to move the track in order to successively translate the plurality of samples, one at a time, through an illumination region on stage 806, whereon incident light 824 impinges. Stage 806 can further include translation axes and mechanisms for translating sample 808 relative to a fixed position of illumination stage 806. The translation mechanisms can be manually operated (e.g., threaded rods) or can be automatically movable via electrical actuation (e.g., motorized drivers, piezoelectric actuators).

In response to incident light 824, emitted light 826 emerges from sample 808. Emitted light 826 can be generated in a number of ways. For example, in some embodiments, emitted light 826 corresponds to a portion of incident light 824 transmitted through sample 808. In other embodiments, emitted light 826 corresponds to a portion of incident light 824 reflected from sample 808. In yet further embodiments, incident light 824 can be absorbed by sample 808, and emitted light 826 corresponds to fluorescence emission from sample 808 (e.g., from fluorescent components in sample 808) in response to incident light 824. In still further embodiments, sample 808 can be luminescent, and may produce emitted light 826 even in the absence of incident light 824. In some embodiments, emitted light 826 can include light produced via two or more of the foregoing mechanisms.

Light collecting optics 810 are positioned to received emitted light 826 from sample 808. Light collecting optics 810 can be configured to collimate emitted light 826 when light 826 is divergent, for example. Light collecting optics 810 can also be configured to spectrally filter emitted light 826. Filtering operations can be useful, for example, in order to isolate a portion of emitted light 826 arising via one of the mechanisms discussed above from light arising via other processes. For example, the methods described herein are used to determine accurate estimates of the fluorescence spectra of one or more labeling moieties in a sample. Light collecting optics 810 can be configured to filter out non-fluorescence components of emitted light 826 (e.g., components corresponding to transmitted and/or reflected incident light). Further, light collecting optics 810 can be configured to modify the spatial and/or temporal properties of emitted light 826 for particular purposes in embodiments. Light collecting optics 810 transform emitted light 826 into output light 828 which is incident on detector 812.

Detector 812 includes one or more elements such as CCD sensors configured to detect output light 828. In some embodiments, detector 812 can be configured to measure the spatial and/or temporal and/or spectral properties of light 828. Detector 812 generates an electrical signal that corresponds to output light 828, and is communicated via electrical communication line 830 to electronic control system 814.

Electronic control system 814 includes a processor 816, a display device 818, and a user interface 820. In addition to receiving signals corresponding to output light 828 detected by detector 812, control system 814 sends electrical signals to detector 812 to adjust various properties of detector 812. For example, if detector 812 includes a CCD sensor, control system 814 can send electrical signals to detector 812 to control the exposure time, active area, gain settings, and other properties of the CCD sensor.

Electronic control system 814 also communicates with light source 802, light conditioning optics 804, illumination stage 806, and light collecting optics 810 via electrical communication lines 832, 834, 836, and 838, respectively. Control system 814 provides electrical signals to each of these elements of system 800 to adjust various properties of the elements. For example, electrical signals provided to light source 802 can be used to adjust the intensity, wavelength, repetition rate, or other properties of light 822. Signals provided to light conditioning optics 804 and light collecting optics 810 can include signals for configuring properties of devices that adjust the spatial properties of light (e.g., spatial light modulators) and for configuring spectral filtering devices, for example. Signals provided to illumination stage 806 can provide for positioning of sample 808 relative to stage 806 and/or for moving samples into position for illumination on stage 806, for example.

Control system 814 includes a user interface 820 for displaying system properties and parameters, and for displaying captured images of sample 808. User interface 820 is provided in order to facilitate operator interaction with, and control over, system 800. Processor 816 includes a storage device for storing image data captured using detector 812, and also includes computer software that embodies instructions to processor 816 that cause processor 816 to carry out control functions, such as those discussed above for example. Further, the software instructions cause processor 816 to mathematically manipulate the images captured by detector 812 and to carry out the steps of decomposing images obtained by system 800 into contributions from particular labeling species in the sample.

In some embodiments, light conditioning optics 804 include an adjustable spectral filter element such as a filter wheel or a liquid crystal spectral filter. The filter element can be configured to provide for illumination of the sample using different light wavelength bands. Light source 802 can provide light 822 having a broad distribution of spectral wavelength components. A selected region of this broad wavelength distribution is allowed to pass as incident light 824 by the filter element in light conditioning optics 804, and directed to be incident on sample 808. Subsequently, the wavelength of the filter pass-band in light conditioning optics 804 is changed to provide incident light 824 having a different wavelength. Spectrally-resolved images can also be recorded by employing a light source 802 having multiple source elements generating light of different wavelengths, and alternately turning the different source elements on and off to provide incident light 684 having different wavelengths.

Light collecting optics 810 can include configurable spectral filter elements similar to those discussed above in connection with light conditioning optics 804. Therefore, spectral resolution can be provided on the excitation side of sample 808 (e.g., via light conditioning optics 804) and on the emission side of sample 808 (e.g., via light collecting optics 810).

The result of collecting multiple, spectrally resolved images of a sample is an “image stack” where each image in the stack is a two-dimensional image of the sample corresponding to a particular wavelength. Conceptually, the set of images can be visualized as forming a three-dimensional matrix, where two of the matrix dimensions are the spatial length and width of each of the images, and the third matrix dimension is the spectral index. For this reason, the set of spectrally resolved images can be referred to as a “spectral cube” of images. As used herein, a “pixel” in such a set of images (or image stack or spectral cube), refers to a common spatial location for each of the images. Accordingly, a pixel in a set of images includes a value associated with each image at the spatial location corresponding to the pixel.

To isolate contributions from each of multiple labeling agents and/or optical labels in a sample to the image information contained in a multispectral image stack, spectral unmixing methods can be used. Spectral unmixing is a technique that quantitatively separates contributions in an image that arise from spectrally different sources. For example, a sample may contain three different spectral sources. The three different spectral sources may each have different absorption spectra. Typically, the individual absorption spectra of the spectral sources are known before they are used, or they can be measured. Images of the sample under illumination will contain, in the most general case, spectral contributions from each of the three spectral sources.

Spectral unmixing decomposes one or more images that include contributions from multiple spectral sources into a set of component images (the “unmixed images”) that correspond to contributions from each of the spectral sources within the sample. Thus, if the sample includes three different spectral sources (e.g., three different labeling agents and/or optical labels), then an image of the sample can be separated into three unmixed images, each unmixed image reflecting contributions principally from only one of the spectral sources.

The unmixing procedure essentially corresponds to decomposing an image into a set of spectral eigenstates. In many embodiments, the eigenstates are known beforehand, as discussed above. In other embodiments, the eigenstates can sometimes be determined using techniques such as principal component analysis. In either case, once the eigenstates have been identified, an image can be decomposed by calculating a set of values, usually as a coefficient matrix, that corresponds to the relative weighting of each of the eigenstates in the overall image. The contributions of each of the individual eigenstates can then be separated out to yield the unmixed image set.

As an example, a series of two dimensional images having x and y coordinates can be measured for a sample by illuminating the sample at a set of different excitation wavelengths μₖ. As described above, the two dimensional images can be combined to form a three-dimensional image cube I (x,y,k) where the first two indices of the image cube represent coordinate directions, and the third index is a spectral index corresponding to the wavelength of the illumination light. Assuming, for the sake of simplicity, that each of the images of the sample contains spectral contributions from two different spectral sources F(λₖ) and G(λₖ), then the values in the three-dimensional image cube I (x,y,k) may be given by

S(x,y,k)=a(x,y)·F(λₖ)+b(x,y)·G(λₖ) (1)

where 2k is used to denote a given wavelength (or wavelength band). The functions a(x,y) and b(x,y) describe the spatial abundance of the spectral contributions from the two different spectral sources in the sample.

According to Equation (1), the net signal any position in the three-dimensional image cube (i.e., at any two-dimensional pixel coordinate, and at a particular illumination wavelength) is the sum of two contributions, weighted by the relative abundance of each. This can be expressed as

I(λₖ)=aF(λₖ)+bG(λₖ) (2)

The functions F and G can be termed the “spectral eigenstates” for the system because they correspond to the pure spectra for the spectral sources in the sample, which are combined in varying proportions to produce the measured spectral images of the sample. Thus, the sample spectrum is a weighted superposition corresponding to separate contributions from the two spectral sources.

If the spectra F(λₖ) and G(λₖ) are known (or can be deduced), then Equation (2) can be inverted to solve for a and b, provided that spectrum I includes at least two elements (i.e., provided that one has data for at least two wavelengths λₖ). Equation (2) can be rewritten in matrix form as I=EA, so that

A=E⁻¹I (3)

where A is a column vector with components a and b, and E is a matrix whose columns are the spectral eigenstates, namely [F G].

Using Equation (3), measured spectral images of a sample can be used to calculate contributions to the images arising purely from source F and purely from source G at particular pixel locations. The process can be repeated for each pixel location on a selected image (i.e., throughout the range of values x and y in I) to produce an image of the sample that includes contributions only from source F, and another image of the sample that includes contributions only from source G.

In the above discussion, the number of spectral sources is two (i.e., F and G). In general, however, unmixing techniques are not restricted to any particular number of sources. For example, a sample can generally contain m different spectral sources. If the number of wavelengths at which data is collected is n—that is, k=1 . . . n—then matrix E is an n×m matrix instead of an n×2 matrix, as in the above discussion. The unmixing algorithm can then be employed in the same manner as described above to isolate specific contributions of spectral sources at each pixel location in an image from each of the m spectral eigenstates.

One factor which can limit the ability of the algorithm to distinguish between contributions from different spectral eigenstates is the degree of spectral distinction between the eigenstates. The correlation between two spectra, such as two spectral eigenstates I₁ and I₂, can be described by a spectral angle θ where

$$θ = {cos}^{- 1} [ \frac{{I}_{1} \cdot {I}_{2}}{\vert {I}_{1} \vert \vert {I}_{2} \vert} ] \qquad ( 4 )$$

Sets of spectra for which θ is small for two members are not as easily separated into their components. Physically, the reason for this is easily understood: if two spectra are only marginally different, it is harder to determine the relative abundance of each.

A number of techniques can be used to measure or estimate the pure spectra of the spectral sources F and G (and other spectral sources, where the sample includes more than two). In general, any method that yields spectral eigenstates of sufficient accuracy can be used. Some samples can contain spectral sources such as dyes or other chemical moieties for which there are known spectra available in published reference materials. Alternatively, it may be possible to directly measure the spectra of source components using one or more measurement systems. In some samples, a particular region of the sample may be known to include only one particular spectral source, and the spectrum of that source can be extracted from measurements taken on only the identified region of the sample.

Various data analysis techniques can also be used for determining component spectra for spectral unmixing, such as principal component analysis (PCA), which identifies the most orthogonal spectral eigenvectors from an image cube and yields score images showing the weighting of each eigenvector throughout the image. This may be done in combination with other mathematical processing, and there are other known techniques for identifying low-dimensionality spectral vectors, such as projection pursuit, a technique described, for example, in L. Jimenez and D. Landgrebe, “Hyperspectral Data Analysis and Feature Reduction Via Projection Pursuit”, IEEE Transactions on Geoscience and Remote Sensing, Vol. 37, No. 6, pp. 2653-2667, November 1999, the entire contents of which are incorporated herein by reference. Other techniques include independent component analysis (ICA) and end-member detection algorithms, for example.

These techniques are typically not well-suited to the applications in the life sciences. For example, some techniques are optimized for spectral imaging data sets that contain spectra with dense spectral shapes and well-defined narrow peaks. In some techniques the spectral ranges are large compared to the individual spectral features and peaks that are used for analysis. The presence of peaks, or the ratio of peaks may be then used to classify “end-members” to be separated. Unfortunately, the components in biological samples typically do not have such well-defined, narrow peaks.

Some of these techniques generate images related to spectra that are present in a pure form somewhere within the original image cube. In many cases in the life sciences, signal spectra present in the image cube are mixtures of components. If the component of interest is not in a pure form somewhere in the original image cube, then it is unlikely that these techniques will generate an image that accurately represents the abundance of the component of interest.

There are some techniques, sometimes called “convex-hull” algorithms, that estimate what the true end-members are even if they do not exist in a pure form in the image, but the effectiveness is dependent on how close signal spectra in the image cube are to the end-members.

One technique that can be used to extract spectral eigenstates (or representations thereof) without a priori knowledge of all of the eigenstates involves considering the signal spectrum I (λₖ) for a given pixel, and subtracting from it the maximum amount of a first spectral source F (λₖ) while leaving the remaining signal that is positive definite in all spectral channels. That is, one defines a so-called “remainder spectrum” Uₐ(λₖ) for each pixel as

Uₐ(λₖ)=I(λₖ)−aF(λₖ) (5)

and then selects the largest value of the parameter a consistent with Uₐ(λₖ) having a non-negative value in every spectral channel. The resulting spectrum Uₐ(λₖ) is then used as the signal spectrum, expunged of contributions due to first spectral source F. One may also make the determination of parameter a based not on strict non-negative criterion listed above, but on some related criteria that incorporates a small negative distribution, to account for considerations such as shot noise or detector noise in a measurement system. Additional examples of optimization criteria for removing the maximal amount of spectral source F include using different error functions.

Alternatively, one may seek to extract a contribution to a measured spectrum that is due to second spectral source G. In analogy with Equation (5), the remainder spectrum can be calculated for each pixel as

U_{b}(λₖ)=I(λₖ)−bG(λₖ) (6)

where one selects the largest value of the parameter b consistent with U_{b}(λₖ) having a non-negative value in every spectral channel.

The remainder technique can be expanded to cases where the spectra for one or more additional components of the sample are known, and one wants to remove their contributions to the signal. In such cases, the remainder spectrum is written to subtract a contribution of each such component from the observed signal based on the additional spectra and consistent with a positive remainder in each spectral channel.

Additional aspects of spectral unmixing are described in U.S. Pat. Nos. 10,126,242 and 7,555,155, and in PCT Patent Publication No. WO2005/040769, the entire contents of each of which are incorporated herein by reference.

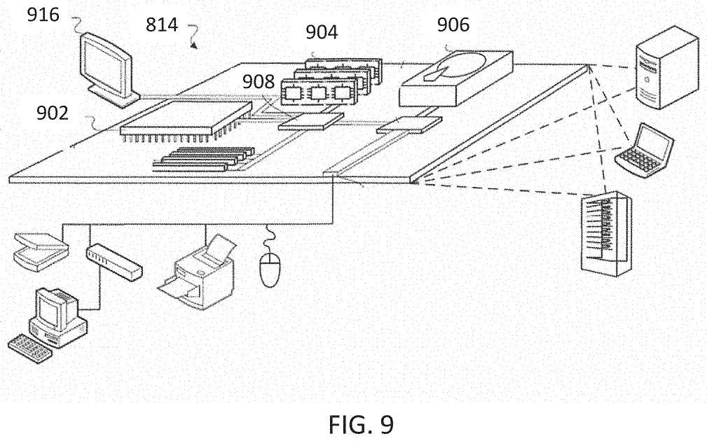

FIG. 9 shows an example of an electronic control system 814, which may be used with the systems and methods disclosed herein. Electronic control system can include one or more processors 902 (e.g., corresponding to processor 816 in FIG. 8), memory 904, a storage device 906 and interfaces 908 for interconnection. The processor 902 can process instructions for execution within the electronic control system 814, including instructions stored in the memory 904 or on the storage device 906. For example, the instructions can instruct the processor 902 to perform any of the analysis and control steps disclosed herein.

The memory 904 can store executable instructions for processor 902, information about parameters of the system such as excitation and detection wavelengths, and measured spectral image information. The storage device 906 can be a computer-readable medium, such as a floppy disk device, a hard disk device, an optical disk device, or a tape device, a flash memory or other similar solid state memory device, or an array of devices, including devices in a storage area network or other configurations. The storage device 906 can store instructions that can be executed by processor 902 described above, and any of the other information that can be stored by memory 904.

In some embodiments, electronic control system 814 can include a graphics processing unit to display graphical information (e.g., using a GUI or text interface) on an external input/output device, such as display 916. The graphical information can be displayed by a display device (e.g., a CRT (cathode ray tube) or LCD (liquid crystal display) monitor) for displaying any of the information, such as measured and calculated spectra and images, disclosed herein. A user can use input devices (e.g., keyboard, pointing device, touch screen, speech recognition device) to provide input to the electronic control system 814.

The methods disclosed herein can be implemented by electronic control system 814 (and processors 902 and 816) by executing instructions in one or more computer programs that are executable and/or interpretable on the electronic control system 814. These computer programs (also known as programs, software, software applications or code) include machine instructions for a programmable processor, and can be implemented in a high-level procedural and/or object-oriented programming language, and/or in assembly/machine language. For example, computer programs can contain the instructions that can be stored in memory 904, in storage unit 906 , and/or on a tangible, computer-readable medium, and executed by processor 902 (processor 816) as described above. As used herein, the term “computer-readable medium” refers to any computer program product, apparatus and/or device (e.g., magnetic discs, optical disks, memory, Programmable Logic Devices (PLDs), ASICs, and electronic circuitry) used to provide machine instructions and/or data to a programmable processor, including a machine-readable medium that receives machine instructions.

Generally, electronic control system 814 can be implemented in a computing system to implement the operations described above. For example, the computing system can include a back end component (e.g., as a data server), or a middleware component (e.g., an application server), or a front end component (e.g., a client computer having a graphical user-interface), or any combination thereof.

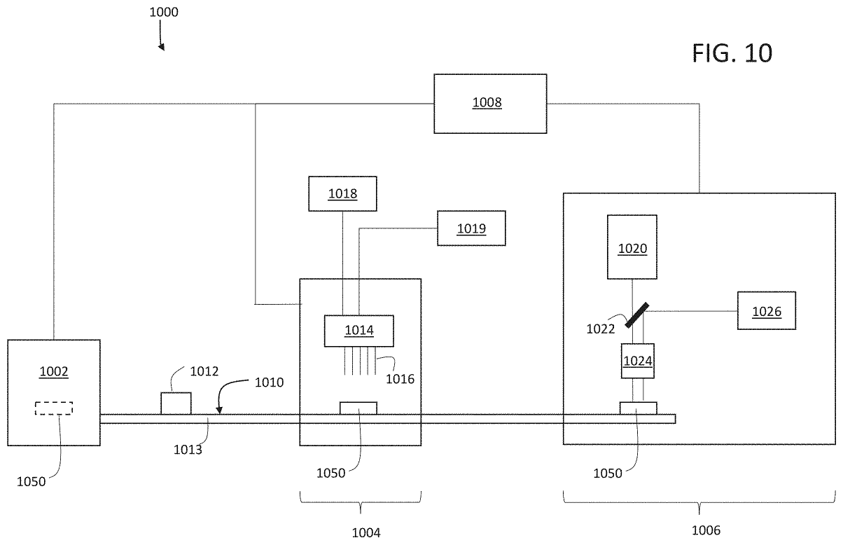

The methods described herein can be implemented in a variety of different analysis systems, including systems that perform some or all of the steps in semi-automated or fully automated fashion. One example of such a system 1000 is shown schematically in FIG. 10. System 1000 includes a storage unit 1002, a labeling station 1004, an imaging station 1006, and a translation apparatus 1010. Each of these components is connected to controller 1008, which includes one or more electronic processors that perform control functions associated with any of the steps and/or analysis functions described herein. In some embodiments, imaging station 1006 corresponds to system 800 shown in FIG. 8. Alternatively, imaging station 1006 can differ from system 800, and can include additional components and/or certain components of system 800 may not be present in imaging station 1006. In certain embodiments, controller 1008 corresponds to electronic control system 814 shown in FIGS. 8 and 9. Alternatively, controller 1008 can differ from electronic control system 814, can include additional components not shown in connection with electronic control system 814, and/or certain components of electronic control system 814 may not be present in controller 1008. In general, controller 1008 can include any of the components shown in connection with electronic control system 814.

Translation apparatus 8010 includes a slide handler 1012 that attaches to individual slides on which biological samples are disposed for analysis, and transport the slides between different locations in the system. An example of a slide on which a sample is disposed is indicated as slide 1050 in FIG. 10. Slide handler 1012 can be implemented in a number of ways. In some embodiments, for example, slide handler 1012 is a grasper and includes one or more arms or fingers that exert pressure on surfaces of slide 1050 to lift and transport slide 1050. In certain embodiments, slide handler 1012 includes a member with one or more suction ports that uses reduced pressure to lift individual slides 1050. In certain embodiments, slide handler 1012 includes one or more members that are inserted under slides 1050 to lift the slides. In general, slide handler 1012 can permit both rotational displacements of slides 1050 about three orthogonal axes, and translations along three orthogonal axes.

Translation apparatus 1010 can also include a track or conveyor 1013 that carries individual slides 1050 or containers of slides between locations in system 1000. In some embodiments, track 1013 is a linear track that moves back and forth along a single direction between locations. In certain embodiments, track 1013 is a continuous track (e.g., circular, elliptical, or another continuous shape) that circulates among locations in the system.

During operation, controller 1008 can transmit appropriate control signals to translation apparatus 1010 to retrieve one or more slides 1050 from storage unit 1002, and to deposit one or more slides into storage unit 1002, as discussed above. Further, controller 1008 can transmit control signals to translation apparatus 1010 to activate labeling station 1004 to deliver fluids, reagents, and compositions to slide 1050, and remove fluids, reagents, compositions (and components thereof) from slide 1050. Labeling station 1004 includes a fluidic apparatus 1014 connected to one or more reservoirs 1018 and to one or more pumps and/or vacuum sources 1019. The fluidic apparatus 1014 includes one or more fluid conduits (e.g., syringes, tubes, pipettes) are coupled to the one or more reservoirs 1018, and are optionally positionable relative to slide 1050, to deliver any of the reagents and/or compositions disclosed herein to the biological sample disposed on slide 1050. Fluid conduits that are optionally positionable can be formed from flexible conduit materials such as any one or more of various conventional polymer materials, and coupled to robotic actuators (not shown in FIG. 10) that are connected to, and receive positioning instructions from, controller 1008.

During operation of the system, controller 1008 transmits signals to translation apparatus 1010 to position slide 1050 within labeling station 1004, and transmits signals to the fluidic apparatus 1014 to cause one or more of the fluid conduits to deliver fluids, reagents, and/or compositions from reservoirs 1018 to the biological sample disposed on slide 1050. Further controller 1008 transmits signals to activate the one or more pumps and/or vacuum sources 1019 to selectively remove fluids, reagents, and/or compositions (and components thereof) from the biological sample. In this manner, controller 1008 (transmitting instructions to labeling station 1004) can implement any of the steps described herein in automated fashion.

Imaging station 1006 includes a radiation source 1020, an objective lens 1024, a beam splitter 1022, and an image detector 1026. Radiation source 1020 can include any one or more of a variety of different sources, including but not limited to LEDs, laser diodes, metal halide sources, incandescent sources, and fluorescent sources. Image detector 1026 can include one or more different detector types, including but not limited to CCD detectors and CMOS detectors. During operation of system 1000, to obtain an image of a biological sample disposed on slide 1050, controller 1008 activates translation apparatus 1010 to position slide 1050 within imaging station 1006. Controller 1008 then transmits control signals to the components of the imaging station, activating source 1020 to deliver illumination radiation to the sample which passes through beam splitter 1022 and objective lens 1024 and is incident on the sample. Emitted light from the sample passes through objective lens 1024, is reflected from beam splitter 1022, and is incident on image detector 1026, which measures an image of the emitted light.

In FIG. 10, the imaging station is configured to obtain a fluorescence or reflected-light image of the sample. However, it should be appreciated that in some embodiments, detector 1026 can be positioned on an opposite side of slide 1050 from source 1020 to measure a transmitted-light image of the sample. In certain embodiments, imaging station 1006 includes multiple detectors for measuring both transmitted- and reflected- or emitted-light (e.g., fluorescence) images of the sample. Under the control of controller 1008, imaging station 1006 can obtain any of the different types of images corresponding to any of the different types of dyes, stains, probes, and labeling agents described herein.

Further, it should be noted that while in the example system 1000 of FIG. 10 the labeling station 1004 and imaging station 1006 are separate, in some embodiments the labeling and imaging stations can be combined into a single station under the control of controller 1008, and which performs both sample labeling and imaging functions as described herein.

Each of the steps in sample analysis consumes a certain amount of time, and improved efficiency can be obtained by translating multiple slides 1050 among multiple locations in system 1000, and performing certain operations in parallel. For example, during analysis of multiple slides 1050, a first slide on which a first sample is disposed can be positioned at the labeling station, and the fluidic apparatus can deliver one or more probes to the sample and incubate the sample with the delivered probes. At the same time, a second slide on which a second sample is disposed can be positioned at the imaging station to obtain one or more images of the sample. A third slide on which a third sample is disposed can be positioned at the labeling station, where controller 1008 activates the one or more pumps and/or vacuum sources 1019 to remove fluids, reagents, and/or compositions (and components thereof) from the sample disposed on the third slide. A fourth slide on which a fourth sample is disposed can be positioned at the labeling station, where controller 1008 activates the fluidic apparatus 1014 to deliver one or more fluids, reagents, and/or compositions, and incubates the fourth sample with the fluids, reagents, and/or compositions. Other slides 1050 can also be processed—undergoing any of the operations discussed herein—at the same time.

As each slide reaches the end of a set of one or more steps in a preparation or analysis workflow, the slide is transported by translation apparatus 1010 to a different location in the system—to a different station, or to a different location within the same station, for example. In this manner, system 1000 can analyze multiple samples in parallel, ensuring that the duty cycles of the components of system 1000 remain relatively high, increasing overall throughput of the system for multiple samples relative to simple linear processing of individual samples.

Additional aspects and features of system 1000 are described in U.S. Patent Application Publication No. US 2020/0393343, the entire contents of which are incorporated herein by reference.

In general, controller 808 can by configured by software instructions, hardward instructions and/or components, or a combination of software and hardware, to perform any of the methods and steps described herein. A user of system 1000 can provide a variety of different types of instructions and information to controller 1008 via input devices. The instructions and information can include, for example, information about any of the parameters (e.g., dyes, labels, probes, reagents, conditions) associated with any of the workflows described herein, calibration information for quantitative analysis of sample images, and instructions following manual analysis of sample images by a technician. Controller 1008 can use any of these various types of information to perform the methods and functions described herein. It should also be noted that any of these types of information can be stored (e.g., in a storage device) and recalled when needed by controller 1008.

By executing software and/or hardware instructions as described above (which can optionally be part of controller 1008), the controller can be configured to implement any one or more of the various steps described in connection with any of the workflows herein. For example, controller 1008 can selectively direct particular fluids, reagents, and/or compositions to particular regions of a biological sample (e.g., by activating fluidic apparatus 1014), and selectively remove fluids, reagents, and/or compositions (and components thereof) from the sample. Controller 1008 can select particular probes (with particular reporter moieties) to deliver to the biological sample, and select particular optical labels (including combinations of optical labels, according to the criteria described above) for delivery to the sample. Controller 1008 can obtain measurements of optical signals from optical labels in the sample, and determine locations of the optical signals based on acquired images.

Controller 1008 can determine the locations of particular analytes in the sample based on the locations of the optical signals and the types of optical labels delivered to the sample in one or more cycles of label exposure and imaging. For example, during each cycle of a workflow, controller 1008 can identify particular label regions present at particular locations in the sample. Based on combinations of label regions present at particular locations in the sample, controller 1008 (e.g., by comparing to stored information about combinations of label regions in specific types of probes) can identify particular analytes at locations in the sample. Controller 1008 can also implement error correction steps as described herein in circumstances where measured signals from optical labels cannot be unambiguously used to identify particular analytes at locations in the sample.

### OTHER EMBODIMENTS

While this disclosure describes specific implementations, these should not be construed as limitations on the scope of the disclosure, but rather as descriptions of features in certain embodiments. Features that are described in the context of separate embodiments can also generally be implemented in combination in a single embodiment. Conversely, various features that are described in the context of a single embodiment can also be implemented in multiple embodiments separately or in any suitable sub-combination. Moreover, although features may be described above as present in certain combinations and even initially claimed as such, one or more features from a claimed combination can generally be excised from the combination, and the claimed combination may be directed to a sub-combination or variation of a sub-combination.

In addition to the embodiments expressly disclosed herein, it will be understood that various modifications to the embodiments described may be made without departing from the spirit and scope of the disclosure. Accordingly, other embodiments are within the scope of the following claims.

1. A method, comprising:
(a) contacting a biological sample comprising a target RNA with a probe, wherein the probe comprises a capture moiety that specifically binds to the target RNA, and a plurality of reporter moieties; and
(b) for each reporter moiety of the plurality of reporter moieties:
contacting the reporter moiety with a catalytic agent comprising an oligonucleotide that specifically binds to the reporter moiety, and a reactive species linked to the oligonucleotide; and
contacting the biological sample with a labeling agent that reacts with the reactive species to deposit the labeling agent or a derivative thereof in the sample in proximity to the target RNA.

2. The method of claim 1, wherein the probe comprises at least three different reporter moieties.

3. (canceled)

4. The method of claim 1, wherein the catalytic agent comprises an enzyme.

5. (canceled)

6. The method of claim 4, wherein the labeling agent comprises an enzyme substrate.

7. (canceled)

8. The method of claim 6, wherein the labeling agent comprises an enzyme substrate conjugated to an oligonucleotide.

9. The method of claim 1, further comprising repeating steps (a) and (b) for additional target RNAs in the biological sample.

10. The method of claim 9, wherein for each different type of target RNA in the biological sample, a unique combination of different labeling agents is deposited in the biological sample in proximity to target RNA molecules.

11. The method of claim 10, wherein each of the different labeling agents comprises an oligonucleotide comprising a unique sequence.

12. The method of claim 11, further comprising:
(c) exposing the biological sample to a plurality of optical labels, wherein each of the optical labels comprises an oligonucleotide having a sequence, and a species that generates an optical signal;
(d) measuring optical signals generated by optical labels of the plurality of optical labels that hybridize to complementary oligonucleotides of the labeling agents;
(e) repeating steps (c) and (d) with different pluralities of optical labels;
(f) identifying one or more of the unique combinations of different labeling agents in the biological sample based on the measured optical signals; and
(g) determining a location of one or more of the target RNAs in the biological sample based on the identified combinations of different labeling agents.

13. The method of claim 12, further comprising, for at least one of the optical labels, removing the at least one of the optical labels from the biological sample after measuring an optical signal generated by the at least one of the optical labels and before repeating steps (c) and (d) with different pluralities of optical labels.

14. The method of claim 13, wherein removing the at least one of the optical labels comprises dehybridizing the at least one of the optical labels from oligonucleotides of one or more of the labeling agents.

15-17. (canceled)

18. The method of claim 12, wherein measuring optical signals generated by the optical labels comprises obtaining at least one image of the optical labels in the biological sample, and identifying optical signals corresponding to the optical labels in the at least one image.

19. (canceled)

20. The method of claim 12, wherein each plurality of optical labels in step (c) comprises 3 or more different types of optical labels.

21. (canceled)

22. The method of claim 1, further comprising repeating step (c) until the biological sample has been exposed to a set of optical labels, wherein each labeling agent deposited in the biological sample has a complementary optical label in the set of optical labels.

23. The method of claim 12, further comprising exposing the biological sample to at least one of the plurality of optical labels more than once.

24. The method of claim 12, wherein each time step (c) is performed, each member of the plurality of optical labels in step (c) comprises a species that generates a different optical signal.

25. (canceled)

26. The method of claim 12, wherein among the plurality of optical labels:
at least two of the optical labels comprise a common species that generates the optical signal; and
the at least two of the optical labels are exposed to the biological sample during different repetitions of step (c).

27. The method of claim 12, wherein among the plurality of optical labels:
first and second optical labels each comprise a first species that generates the optical signals of the first and second optical labels;
third and fourth optical labels each comprise a second species that generates the optical signals of the third and fourth optical labels; and
the first and second species are different.

28-29. (canceled)

30. The method of claim 1, wherein the labeling agent comprises a species that generates an optical signal.

31. (canceled)

32. The method of claim 30, further comprising obtaining one or more images of the sample comprising contributions from each of the labeling agents, wherein different labeling agents are associated with each of the reporter moieties of the plurality of reporter moieties.

33. (canceled)

34. The method of claim 32, further comprising repeating steps (a) and (b) for multiple different target RNAs in the biological sample.

35. The method of claim 34, further comprising, prior to repeating steps (a) and (b), inactivating at least some of the deposited labeling agents in the sample.

36. (canceled)

37. The method of claim 34, further comprising selecting the labeling agents so that a unique combination of labeling agents is deposited in the biological sample in proximity to each different target RNA.

38-42. (canceled)

43. The method of claim 32, wherein the one or more images comprise contributions from labeling agents associated with more than one type of target RNA in the biological sample.

44. A method, comprising:
(a) contacting a biological sample comprising a target RNA with a probe, wherein the probe comprises a capture moiety that specifically binds to the target RNA, and a plurality of reporter moieties;
(b) for each reporter moiety of the plurality of reporter moieties:
contacting the reporter moiety with a catalytic agent comprising an oligonucleotide that specifically hybridizes to the reporter moiety, and a reactive species linked to the oligonucleotide; and
contacting the biological sample with a labeling agent comprising a substrate conjugated to an optical label, wherein the substrate reacts with the reactive species to deposit the labeling agent or a derivative thereof in the biological sample in proximity to the target RNA;
(c) measuring optical signals generated by optical labels of each of the deposited labeling agents in the biological sample; and
(d) determining a location of the target RNA in the biological sample based on the measured optical signals.

45. A method, comprising:
(a) contacting a biological sample comprising different target RNAs with a plurality of different probes, wherein each different probe selectively hybridizes to a different target RNA and comprises a different combination of a plurality of reporter moieties;
(b) contacting the biological sample with a catalytic agent, wherein the catalytic agent comprises an oligonucleotide that specifically hybridizes to one type of reporter moiety, and a reactive species linked to the oligonucleotide;
(c) contacting the biological sample with a labeling agent comprising a substrate conjugated to a labeling oligonucleotide, wherein the substrate reacts with the reactive species to deposit the labeling oligonucleotide in the biological sample in proximity to the one type of reporter moiety;
(d) repeating steps (b) and (c) with different catalytic agents, wherein each of the different catalytic agents comprises an oligonucleotide that specifically hybridizes to one type of reporter moiety;
(e) contacting the biological sample with a plurality of optical labels each comprising a reporter oligonucleotide conjugated to an optical moiety, wherein each reporter oligonucleotide hybridizes to one type of labeling oligonucleotide;
(f) measuring optical signals generated by optical labels in the biological sample; and
(g) determining a location of one or more target RNAs in the biological sample based on the measured optical signals.
